(19) United States
(12) Patent Application Publication
(10) Pub. No.: US 2014/0248278 A1
(43) Pub. Date: Sep. 4, 2014

(54) ANTI-CD22 ANTIGEN BINDING MOLECULES TO TREAT LUNG CANCER AND PROSTATE CANCER

(71) Applicants: Joseph Tuscano, Folsom, CA (US); Robert O'Donnell, Sacramento, CA (US)

(72) Inventors: Joseph Tuscano, Folsom, CA (US); Robert O'Donnell, Sacramento, CA (US)

(73) Assignee: The Regents of the University of California, Oakland, CA (US)

(21) Appl. No.: 14/123,756

(22) PCT Filed: Jun. 8, 2012
(86) PCT No.: PCT/US2012/041500

§ 371 Date: May 19, 2014

Related U.S. Application Data
(60) Provisional application No. 61/494,758, filed on Jun. 8, 2011.

(51) Int. Cl. C07K 16/30

(52) U.S. Cl. CPC C07K 16/3023
USPC 424/144.1; 435/375; 435/366

(57) ABSTRACT

This invention provides methods for preventing, reducing, delaying or inhibiting the proliferation and/or growth and/or metastasis of lung cancers and prostate cancer that express or overexpress CD22 by contacting the lung cancer cell or prostate cancer cell with an antigen binding molecule that binds to CD22 expressed on the surface of the cancer cell.

### FIELD OF THE INVENTION

This application is a U.S. national phase under 35 U.S.C. §371 of International Application No. PCT/US2012/041500, filed on Jun. 8, 2012, which claims the benefit under 35 U.S.C. §119(e) of U.S. Provisional Application No. 61/494,758, filed on Jun. 8, 2011, both of which are hereby incorporated herein by reference in their entirety for all purposes.

### FIELD OF THE INVENTION

The present invention relates to methods of preventing, reducing, delaying and inhibiting the proliferation and/or growth and/or metastasis of lung cancer and/or prostate cancer cells by contacting the cancer cell with an antigen binding molecule, e.g., a peptide, a non-antibody binding molecule, an antibody or antibody fragment, that binds to CD22.

### BACKGROUND OF THE INVENTION

In the United States, lung cancer is the most common cause of cancer-death in both men and women (Minna J D. “Neoplasms of the Lung,” In: Kasper D L, editor. Harrison's Principles of Internal Medicine. 16th ed; 2005. p. 506-515). Despite refinements in platinum-based chemotherapy and several newly approved targeted agents, the median overall survival of patients with advanced, unresectable, NSCLC is only 8-11 months (Detterbeck, et al., Chest (2009) 136(1):260-71; Wang, et al., Cancer (2010) 116(6):1518-25). MAbs and tyrosine kinase inhibitors (TKIs) that inhibit signaling from epidermal growth factor receptor (EGFR) and vascular endothelial cell growth factor (VEGF) provide clinical benefit to some NSCLC patients. Still, the EGFR inhibitor erlotinib is effective in only a small subset of patients and they inevitably develop resistance; the VEGF-targeted mAb bevacizumab adds only incrementally to progression-free survival (Sun, et al., J Clin Invest (2007) 117(10):2740-50; Stinchcombe, et al., Proc Am Thorac Soc (2009) 6(2):233-41; and Katzel, et al., J Hematol Oncol (2009) 2:2). New therapeutic approaches are essential if significant advances are to be made in the treatment of NSCLC. The present invention is based, in part, on the discovery of CD22 as a target on NSCLC. Several anti-CD22 monoclonal antibodies (mAb) have been found to bind to lung and prostate cancer including those developed for the treatment of non-Hodgkin's lymphoma (NHL). Once anti-CD22 mAb, HB22.7, effectively binds NSCLC and mediates specific in vitro and in vivo killing.

CD22 as a target for drug development. CD22 is a 140 kDa single-pass transmembrane sialo-adhesion protein that influences B-cell survival (Tedder, et al., Annu Rev Immunol (1997) 15:481-504; and Tedder, et al., Adv Immunol (2005) 88:1-50). Nearly all mature B-cells express CD22 as do most NHL (Crocker, et al., Nat Rev Immunol (2007) 7(4):255-66; Collins, et al., J Immunol (2006) 177(5):2994-3003; Haas, et al., J Immunol (2006) 177(5):3063-73; and Engel, et al., J Exp Med (1995) 181(4):1581-6). CD22 is a member of the immunoglobulin (Ig) superfamily and possesses seven extracellular Ig-like domains. The two amino-terminal Ig domains of CD22 mediate cell adhesion to widely distributed α(2,6) sialic-acid bearing ligands, and B-cell homing to endothelial cells (Crocker, et al., Nat Rev Immunol (2007) 7(4):255-66).

An important function of CD22 in B-cells is to modulate for B-cell antigen receptor (BCR) signaling. Within the cytoplasmic tail of CD22 are immunoreceptor tyrosine activation motifs (ITAMs) and tyrosine inhibitory motifs (ITIMs) (Tedder, et al., Annu Rev Immunol (1997) 15:481-504; Tedder, et al., Adv Immunol (2005) 88:1-50). CD22 ITAMs become phosphorylated upon BCR activation by src-like kinases, enhancing the recruitment of protein tyrosine phosphatases to CD22. Due to the close proximity of the BCR, these CD22-associated phosphatases then dephosphorylate BCR components resulting in attenuation of BCR signaling. The consequences of BCR-independent engagement of CD22 on B-cell function, as well as the downstream signal transduction events triggered through CD22 have only recently been explored. It has been demonstrated that potent and direct activation of CD22 via ligand binding and crosslinking is cytotoxic for B-cell NHL (Tedder, et al., Annu Rev Immunol (1997) 15:481-504; Tedder, et al., Adv Immunol (2005) 88:1-50; Tuscano, et al., Blood (1999) 94(4):1382-92; Tuscano, et al., Eur J Immunol (1996) 26(6):1246-52; Tuscano, et al., Blood (1996) 87:4723-4730).

It has heretofore been thought that CD22 was exclusively expressed on B-cells. The observation that CD22 is expressed on NSCLC identifies an unexplored mechanism of NSCLC tumorigenesis and moreover provides a method for CD22 antigen-targeted therapy for NSCLC where there are few if any tumor-specific targets.

### SUMMARY OF THE INVENTION

In one aspect, the present invention provides methods of preventing, reducing, delaying or inhibiting the proliferation and/or growth of a lung cancer cell. In some embodiments, the methods comprise contacting the lung cancer cell with an antigen binding molecule that binds to CD22 expressed on the surface of the lung cancer cell.

In another aspect, the invention provides methods of preventing, reducing, delaying or inhibiting the proliferation and/or growth of a prostate cancer cell. In some embodiments, the methods comprise contacting the prostate cancer cell with an antigen binding molecule that binds to CD22 expressed on the surface of the prostate cancer cell.

In a further aspect, the invention provides methods of preventing, reducing, delaying or inhibiting the proliferation and/or growth and/or metastasis of a lung cancer in a subject in need thereof. In some embodiments, the methods comprise administering to the subject an antigen binding molecule that binds to CD22, wherein the antigen binding molecule binds to CD22 expressed on the lung cancer, thereby preventing, reducing, delaying or inhibiting the growth or metastasis of the lung cancer in the subject.

In a related aspect, the invention provides methods of preventing, reducing, delaying or inhibiting the proliferation and/or growth and/or metastasis of a prostate cancer in a subject in need thereof. In some embodiments, the methods comprise administering to the subject an antigen binding molecule that binds to CD22, wherein the antigen binding molecule binds to CD22 expressed on the prostate cancer, thereby preventing, reducing, delaying or inhibiting the growth or metastasis of the prostate cancer in the subject.

With respect to the embodiments, in some embodiments, the antigen binding molecule is a peptide that binds to CD22. In some embodiments, the antigen binding molecule is a non-antibody binding protein. In some embodiments, the antigen binding molecule is an antibody or antibody fragment that binds to CD22. In some embodiments, the antibody or antibody fragment that binds to CD22 is monoclonal. In some embodiments, the anti-CD22 antibody or antibody fragment is HB22.7 (i.e., comprises the minimal binding determinant of HB22.7, e.g., comprises heavy and light chain complementarity determining regions CDR1, CDR2 and CDR3 of HB22.7). In some embodiments, the anti-CD22 antibody or antibody fragment is hHB22.7 (i.e., comprises the minimal binding determinant of HB22.7, e.g., comprises heavy and light chain complementarity determining regions CDR1, CDR2 and CDR3 of HB22.7). In some embodiments, the anti-CD22 antibody or antibody fragment is a human chimera. In some embodiments, the anti-CD22 antibody or antibody fragment is humanized. In some embodiments, the anti-CD22 antibody or antibody fragment is human. In some embodiments, the antigen binding molecule is an IgG antibody. In some embodiments, the IgG antibody is human IgG1 isotype or human IgG3 isotype.

In some embodiments, the antigen binding molecule, or antibody or antibody fragment is conjugated to a therapeutic agent. In some embodiments, the therapeutic agent is selected from the group consisting of a cytotoxin, a radionuclide, an inhibitory nucleic acid, a chemotherapeutic agent and an anti-neoplastic agent. In some embodiments, the therapeutic agent is encapsulated in a liposome or in a nanoparticle. In some embodiments, the antigen binding molecule, or antibody or antibody fragment can be conjugated to or integrated into the liposome or the nanoparticle.

In embodiments where the cancer cell is a lung cancer cell, in some embodiments, the lung cancer cell is a non-small cell lung cancer cell. In some embodiments, the lung cancer cell expresses or overexpresses CD22 on the cell surface.

In embodiments where the cancer cell is a prostate cancer cell, in some embodiments, the prostate cancer cell is hormone sensitive. In some embodiments, the prostate cancer cell is hormone refractory. In some embodiments, the prostate cancer cell expresses or overexpresses CD22 on the cell surface.

In embodiments where the cancer is a lung cancer, in some embodiments, the lung cancer is a non-small cell lung cancer. In some embodiments, the non-small cell lung cancer is a subtype selected from the group consisting of squamous cell, adenocarcinoma, adenosquamous, large cell, bronchioalveolar, carcinoid and mixed tumors of bronchoepithelial origin. In some embodiments, the lung cancer expresses or overexpresses CD22 on the cell surface.

In embodiments where the cancer is a prostate cancer, in some embodiments, the prostate cancer is hormone sensitive. In some embodiments, the prostate cancer is hormone refractory. Any patient with localized or metastatic prostate cancer may be a subject for the use of the CD22-antigen binding molecules, targeting CD22 expressed on prostate tissue. In some embodiments, the prostate cancer expresses or overexpresses CD22 on the cell surface.

In some embodiments, the lung cancer cell or the prostate cancer cell is in vitro. In some embodiments, the lung cancer cell or the prostate cancer cell is in vivo. In some embodiments, the lung cancer cell or the prostate cancer cell is human.

In some embodiments, the subject is a human.

In some embodiments, the subject does not have a hematological cancer. In some embodiments, the subject does not have a B cell malignancy. In some embodiments, the antigen binding molecule, or antibody or antibody fragment is not co-administered with a chemotherapeutic agent or an anti-neoplastic agent. In some embodiments, the subject does not have any disease condition or any cancer other than a lung cancer. In some embodiments, the subject does not have any other disease condition or any cancer other than prostate cancer.

In some embodiments, the antigen binding molecule, or antibody or antibody fragment is administered intravenously or subcutaneously.

### DEFINITIONS

“CD22” refers to a lineage-restricted B cell antigen belonging to the Ig superfamily. It is expressed in 60-70% of B cell lymphomas and leukemias and is not present on the cell surface in early stages of B cell development or on stem cells. See, e.g. Vaickus et al., Crit. Rev. Oncol/Hematol. 11:267-297 (1991). The nucleic acid sequences and encoded amino acid sequences of human CD22 have been assigned GenBank accession numbers NM_{—}001771.3→NP_{—}001762.2 (isoform 1); NM_{—}001185099.1→NP_{—}001172028.1 (isoform 2); NM_{—}001185100.1→NP_{—}001172029.1 (isoform 3); and NM_{—}001185101.1→NP_{—}001172030.1 (isoform 4).

As used herein, the term “anti-CD22” in reference to an antibody, refers to an antibody that specifically binds CD22 and includes reference to an antibody which is generated against CD22. In preferred embodiments, the CD22 is a primate CD22 such as human CD22. In a particularly preferred embodiment, the antibody is generated against human CD22 synthesized by a non-primate mammal after introduction into the animal of cDNA which encodes human CD22.

The terms “systemic administration” and “systemically administered” refer to a method of administering an antigen binding molecule that binds to CD22 to sites in the body, including the targeted site of pharmaceutical action, via the circulatory system. Systemic administration includes, but is not limited to, oral, intranasal, rectal and parenteral (i.e., other than through the alimentary tract, such as intramuscular, intravenous, intra-arterial, transdermal and subcutaneous) administration.

The term “co-administer” and “co-administering” and variants thereof refer to the simultaneous presence of two or more active agents in the blood of an individual. The active agents that are co-administered can be concurrently or sequentially delivered. In the treatment and prevention of lung cancer and/or prostate cancer, an antigen binding molecule that binds to CD22 can be co-administered with another active agent efficacious in treating or preventing cancer (e.g., a chemotherapeutic agent, an anti-neoplastic agent, an inhibitory nucleic acid, a cytotoxin, etc.).

The phrase “cause to be administered” refers to the actions taken by a medical professional (e.g., a physician), or a person controlling medical care of a subject, that control and/or permit the administration of the agent(s)/compound(s) at issue to the subject. Causing to be administered can involve diagnosis and/or determination of an appropriate therapeutic or prophylactic regimen, and/or prescribing particular agent(s)/compounds for a subject. Such prescribing can include, for example, drafting a prescription form, annotating a medical record, and the like.

The terms “consisting essentially of” and variants thereof refer to the genera or species of active agents expressly identified in a method or composition, as well as any excipients inactive for the intended purpose of the methods or compositions.

The terms “treating” and “treatment” and variants thereof refer to delaying the onset of, retarding or reversing the progress of, alleviating or preventing either the disease or condition to which the term applies, or one or more symptoms of such disease or condition. Treating and treatment encompass both therapeutic and prophylactic treatment regimens.

The terms “inhibiting,” “reducing,” “decreasing” with respect to tumor or cancer growth or progression refers to inhibiting the growth, spread, metastasis of a tumor or cancer in a subject by a measurable amount using any method known in the art. The growth, progression or spread of a tumor or cancer is inhibited, reduced or decreased if the tumor burden is at least about 10%, 20%, 30%, 50%, 80%, or 100% reduced in comparison to the tumor burden prior to administration of an antigen binding molecule that binds to CD22. In some embodiments, the growth, progression or spread of a tumor or cancer is inhibited, reduced or decreased by at least about 1-fold, 2-fold, 3-fold, 4-fold, or more in comparison to the tumor burden prior to administration of the antigen binding molecule that binds to CD22.

The terms “subject,” “patient,” or “individual” interchangeably refer to any mammal, for example: humans, non-human primates (e.g., chimpanzees, or macaques), domestic mammals (e.g., canine, feline), agricultural mammals (e.g., bovine, equine, ovine, porcine) and laboratory mammals (e.g., mouse, rat, rabbit, hamster, guinea pig).

As used herein, “mammalian cells” includes reference to cells derived from mammals including humans and non-human primates (e.g., chimpanzees, or macaques), domestic mammals (e.g., canine, feline), agricultural mammals (e.g., bovine, equine, ovine, porcine) and laboratory mammals (e.g., mouse, rat, rabbit, hamster, guinea pig). The cells may be cultured in vivo or in vitro.

An “antigen binding molecule,” as used herein, is any molecule that can specifically or selectively bind to an antigen. A binding molecule may include an antibody or a fragment thereof. An anti-CD22 binding molecule is a molecule that binds to the CD22 antigen, such as an anti-CD22 antibody or fragment thereof. Other anti-CD22 binding molecules may also include multivalent molecules, multi-specific molecules (e.g., diabodies), fusion molecules, aptimers, avimers, or other naturally occurring or recombinantly created molecules. Illustrative antigen-binding molecules useful to the present methods include antibody-like molecules. An antibody-like molecule is a molecule that can exhibit functions by binding to a target molecule (See, e.g., Current Opinion in Biotechnology 2006, 17:653-658; Current Opinion in Biotechnology 2007, 18:1-10; Current Opinion in Structural Biology 1997, 7:463-469; Protein Science 2006, 15:14-27), and includes, for example, DARPins (WO 2002/020565), Affibody (WO 1995/001937), Avimer (WO 2004/044011; WO 2005/040229), and Adnectin (WO 2002/032925).

An “antibody” refers to a polypeptide of the immunoglobulin family or a polypeptide comprising fragments of an immunoglobulin that is capable of noncovalently, reversibly, and in a specific manner binding a corresponding antigen. An exemplary antibody structural unit comprises a tetramer. Each tetramer is composed of two identical pairs of polypeptide chains, each pair having one “light” (about 25 kD) and one “heavy” chain (about 50-70 kD), connected through a disulfide bond. The recognized immunoglobulin genes include the κ, λ, α, γ, δ, ε, and μ constant region genes, as well as the myriad immunoglobulin variable region genes. Light chains are classified as either κ or λ. Heavy chains are classified as γ, μ, α, δ, or ε, which in turn define the immunoglobulin classes, IgG, IgM, IgA, IgD, and IgE, respectively. The N-terminus of each chain defines a variable region of about 100 to 110 or more amino acids primarily responsible for antigen recognition. The terms variable light chain (VL) and variable heavy chain (VH) refer to these regions of light and heavy chains, respectively. As used in this application, an “antibody” encompasses all variations of antibody and fragments thereof that possess a particular binding specifically, e.g., for tumor associated antigens. Thus, within the scope of this concept are full length antibodies, chimeric antibodies, humanized antibodies, human antibodies, unibodies, single domain antibodies or nanobodies, single chain antibodies (ScFv), Fab, Fab′, and multimeric versions of these fragments (e.g., F(ab′)₂) with the same binding specificity.

Typically, an immunoglobulin has a heavy and light chain. Each heavy and light chain contains a constant region and a variable region, (the regions are also known as “domains”). Light and heavy chain variable regions contain a “framework” region interrupted by three hypervariable regions, also called “complementarity-determining regions” or “CDRs”. The extent of the framework region and CDRs have been defined. See, Kabat and Wu, SEQUENCES OF PROTEINS OF IMMUNOLOGICAL INTEREST, U.S. Government Printing Office, NIH Publication No. 91-3242 (1991); Kabat and Wu, J Immunol. (1991) 147(5):1709-19; and Wu and Kabat, Mol Immunol. (1992) 29(9):1141-6. The sequences of the framework regions of different light or heavy chains are relatively conserved within a species. The framework region of an antibody, that is the combined framework regions of the constituent light and heavy chains, serves to position and align the CDRs in three dimensional space.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a VH CDR3 is located in the variable domain of the heavy chain of the antibody in which it is found, whereas a VL CDR1 is the CDR1 from the variable domain of the light chain of the antibody in which it is found.

References to “VH” refer to the variable region of an immunoglobulin heavy chain, including an Fv, scFv, dsFv or Fab. References to “VL” refer to the variable region of an immunoglobulin light chain, including of an Fv, scFv, dsFv or Fab.

The phrase “single chain Fv” or “scFv” refers to an antibody in which the variable domains of the heavy chain and of the light chain of a traditional two chain antibody have been joined to form one chain. Typically, a linker peptide is inserted between the two chains to allow for proper folding and creation of an active binding site.

The term “linker peptide” includes reference to a peptide within an antibody binding fragment (e.g., Fv fragment) which serves to indirectly bond the variable domain of the heavy chain to the variable domain of the light chain.

The term “parental antibody” means any antibody of interest which is to be mutated or varied to obtain antibodies or fragments thereof which bind to the same epitope as the parental antibody, preferably with equivalent or higher affinity for the target antigen.

The term “specific binding” is defined herein as the preferential binding of binding partners to another (e.g., a polypeptide and a ligand (analyte), two polypeptides, a polypeptide and nucleic acid molecule, or two nucleic acid molecules) at specific sites. The term “specifically binds” indicates that the binding preference (e.g., affinity) for the target molecule/sequence is at least 2-fold, more preferably at least 5-fold, and most preferably at least 10- or 20-fold over a non-specific target molecule (e.g., a randomly generated molecule lacking the specifically recognized site(s); or a control sample where the target molecule or antigen is absent).

With respect to antibodies of the invention, the term “immunologically specific” “specifically binds” refers to antibodies and non-antibody antigen binding molecules that bind to one or more epitopes of a protein of interest (e.g., CD22), but which do not substantially recognize and bind other molecules in a sample containing a mixed population of antigenic biological molecules.

The term “selectively reactive” refers, with respect to an antigen, the preferential association of an antibody, in whole or part, with a cell or tissue bearing that antigen and not to cells or tissues lacking that antigen. It is, of course, recognized that a certain degree of non-specific interaction may occur between a molecule and a non-target cell or tissue. Nevertheless, selective reactivity, may be distinguished as mediated through specific recognition of the antigen. Although selectively reactive antibodies bind antigen, they may do so with low affinity. On the other hand, specific binding results in a much stronger association between the antibody and cells bearing the antigen than between the bound antibody and cells lacking the antigen. Specific binding typically results in greater than 2-fold, preferably greater than 5-fold, more preferably greater than 10- or 20-fold and most preferably greater than 100-fold increase in amount of bound antibody (per unit time) to a cell or tissue bearing CD22 as compared to a cell or tissue lacking CD22.

The term “immunologically reactive conditions” includes reference to conditions which allow an antibody generated to a particular epitope to bind to that epitope to a detectably greater degree than, and/or to the substantial exclusion of, binding to substantially all other epitopes. Immunologically reactive conditions are dependent upon the format of the antibody binding reaction and typically are those utilized in immunoassay protocols or those conditions encountered in vivo. See, e.g., Harlow & Lane, Using Antibodies, A Laboratory Manual (1998), for a description of immunoassay formats and conditions. Preferably, the immunologically reactive conditions employed in the methods of the present invention are “physiological conditions” which include reference to conditions (e.g., temperature, osmolarity, pH) that are typical inside a living mammal or a mammalian cell. While it is recognized that some organs are subject to extreme conditions, the intra-organismal and intracellular environment normally lies around pH 7 (i.e., from pH 6.0 to pH 8.0, more typically pH 6.5 to 7.5), contains water as the predominant solvent, and exists at a temperature above 0° C. and below 50° C. Osmolarity is within the range that is supportive of cell viability and proliferation.

A “targeting moiety” is the portion of an immunoconjugate intended to target the immunoconjugate to a cell of interest. Typically, the targeting moiety is an antibody, a scFv, a dsFv, an Fab, or an F(ab′)₂.

A “toxic moiety” is the portion of a immunotoxin which renders the immunotoxin cytotoxic to cells of interest.

A “therapeutic moiety” is the portion of an immunoconjugate intended to act as a therapeutic agent.

The term “therapeutic agent” includes any number of compounds currently known or later developed to act as anti-neoplastics, anti-inflammatories, cytokines, anti-infectives, enzyme activators or inhibitors, allosteric modifiers, antibiotics, inhibitor nucleic acids or other agents administered to induce a desired therapeutic effect in a patient. The therapeutic agent may also be a chemotherapeutic agent, an anti-neoplastic agent, a cytotoxin or a radionuclide, where the therapeutic effect intended is, for example, the killing of a cancer cell.

A “detectable label” means, with respect to an immunoconjugate, a portion of the immunoconjugate which has a property rendering its presence detectable. For example, the immunoconjugate may be labeled with a radioactive isotope which permits cells in which the immunoconjugate is present to be detected in immunohistochemical assays.

The term “effector moiety” means the portion of an immunoconjugate intended to have an effect on a cell targeted by the targeting moiety or to identify the presence of the immunoconjugate. Thus, the effector moiety can be, for example, a therapeutic moiety, a toxin, a radiolabel, or a fluorescent label.

The term “immunoconjugate” includes reference to a covalent linkage of an effector molecule to an antibody, antibody fragment or an antigen binding molecule. The effector molecule can be an immunotoxin.

The terms “effective amount” or “amount effective to” or “therapeutically effective amount” includes reference to a dosage of a therapeutic agent sufficient to produce a desired result, e.g., reducing or eliminating tumor burden, inhibiting cell protein synthesis by at least 50%, or killing the cell.

The term “toxin” or “cytotoxin” includes reference to abrin, ricin, gelonin, Pseudomonas exotoxin (PE), diphtheria toxin (DT), botulinum toxin, auristatin E, auristatin F, monomethyl auristatin E (MMAE), monomethyl auristatin F (MMAF), or modified toxins thereof. For example, PE and DT are highly toxic compounds that typically bring about death through liver toxicity. PE and DT, however, can be modified into a form for use as an immunotoxin by removing the native targeting component of the toxin (e.g., domain Ia of PE or the B chain of DT) and replacing it with a different targeting moiety, such as an antibody.

The term “contacting” includes reference to placement in direct physical association.

The terms “conjugating,” “joining,” “bonding” or “linking” refer to making two polypeptides into one contiguous polypeptide molecule. In the context of the present invention, the terms include reference to joining an antibody moiety to an effector molecule (EM). The linkage can be either by chemical or recombinant means. Chemical means refers to a reaction between the antibody moiety and the effector molecule such that there is a covalent bond formed between the two molecules to form one molecule. Biodegradable linkers are also contemplated. See, e.g., Meng, et al., Biomaterials. (2009) 30(12):2180-98; Duncan, Biochem Soc Trans. (2007) 35(Pt 1):56-60; Kim, et al., Biomaterials. (2011) 32(22):5158-66; and Chen, et al., Bioconjug Chem. (2011) 22(4):617-24.

The term “in vivo” includes reference to inside the body of the organism from which the cell was obtained. “Ex vivo” and “in vitro” means outside the body of the organism from which the cell was obtained.

The phrase “malignant cell” or “malignancy” refers to tumors or tumor cells that are invasive and/or able to undergo metastasis, i.e., a cancerous cell.

### BRIEF DESCRIPTION OF THE DRAWINGS

FIGS. 1A-G illustrate CD22 expression in NSCLC. (A) Several NSCLC cell lines were probed with FITC-labeled HB22.7. Ramos and Jurkat cells served as CD22 positive and negative controls, respectively. (B) PCR amplification of CD22 from designated cDNA (Lane 1-Ramos, 2-A549, 3-H1650, 4-H727, 5-A427, 6-CD22 plasmid, 7-BEC. (C) Anti-CD22 immunoblot of whole cell lysates derived from B and T-cells, media, A549, Calu1, and Calu6 NSCLC cell lines, respectively. (D) Human CD22 Northern blot of total RNA extracted from Ramos B-cells, A549, H1650, H727, and A427, lanes 1-5, respectively. Detection was accomplished with a DIG-dUTP labeled DNA probe. (E) IHC of lung cancer patient biopsy specimens stained with anti-CD22 mAb (A254 Biotex) with immunoperoxidase detection and H & E counterstain (60X); (F) Anti-CD22 Western Blot of cell membrane fraction from Ramos, H1650, A549, H727, 293T and Calu1 cells; (G) Anti-CD22 Western Blot of cytoplasmic fraction from Ramos, H1650, A549, H727, Raji, A427 and 293T cells.

FIGS. 2A-C illustrate anti-CD22 mediated cytotoxicity and CD22 internalization. (A) Ligation of CD22 mediates cytotoxicity of NSCLC and NHL B cells. Cells were treated with the anti-CD22 or anti-CD20 mAbs (HB22.7 or rituximab, respectively) (50 ug/ml) for 48 hr then assessed with an MTT assay. (B) Internalization of CD22 was assessed on NSCLC cell lines. The degree of internalization was determined by assessing the cytotoxicity of a carrier protein attached to an anti-mouse mAb (ZAP) compared to a non-cytotoxic control (SAP). HB22.7-ZAP and HB22.7-SAP were assessed in Ramos B-cells (top) and two NSCLC cell lines, A549 and H727 (bottom). This also demonstrates the effectiveness of CD22-targeted antibody drug conjugates (ADC) for the treatment of NSCLC(C) ADCC and CDC assays using A549 cells were done to determine the effect of huHB22.7 on human PBMC- or CDC-mediated cytotoxicity. PBMCs (10:1 PBMC:A549 ratio) were incubated +/− complement (1:10 dilution), or +/− huHB22.7 (50 μg/cc). A549-specific cytotoxicity was assessed with a DELFIA EuTDA cytotoxicity assay and reported as % of control.

FIG. 3 illustrates that HB22.7 effectively targets human A549 xenografts in vivo. Mice bearing A549 or Raji NHL flank xenografts (arrow) received ⁶⁴Cu-DOTA-HB22.7 (50 μCi) for I-PET using a micro-PET scanner. Top: transverse views of mice bearing A549 xenografts. Bottom: transverse images of mice bearing human NHL (Raji) xenografts.

FIGS. 4A-D illustrate that the HB22.7 anti-CD22-blocking mAb is active against NSCLC in vivo: (A) Nude mice bearing human BAC/H1650 xenografts were injected intravenously (iv) (arrow) with: (IgG control, upper curve), or HB22.7 (lower curve) (1.4 mg). (B) Nude mice bearing A549 xenografts were injected iv (arrow) with: PBS (untreated), HB22.7 (1.4 mg) or rituximab, before tumors developed (pretreated) or after tumors had grown (*established). (C) Growth of A549 (NSCLC) and PC-3 (prostate cancer) xenografts. Nude mice were injected intravenously (iv) (arrow) with: PBS (untreated), HB22.7 (1.4 mg) before tumors developed (&, pretreated) or HB22.7 (1.4 mg) after tumors had grown (*, established). (D) Mice bearing A549 xenografts were established in non-irradiated nude mice and treated with HB22.7 as described in (B).

FIGS. 5A-B illustrate that the anti-CD22 mAb HB22.7 prevents the development of lung metastasis and improves survival in an orthotopic model of NSCLC. (A) A549 cells were injected IV with (left) or without (right) HB22.7 (1.4 mg) pre-treatment. The lungs were examined in surviving mice 64 days after injection. (B) Kaplan-Meier survival curve of mice bearing orthotopic/iv A549 NSCLC xenografts; mice were treated with HB22.7 (1.4) and compared to untreated control mice.

FIG. 6 illustrates an MTT assay of A549 cells treated with Doxil (doxorubicin, 50 μg/ml) or pegylated liposomal Doxil coated with HB22.7 (IL-Doxil) (50 μg/ml). Cells were treated for 1 hr, washed, then assayed after 24 hrs. (+) control was treated continuously with Doxil.

FIG. 7 illustrates that tumor growth in A549-bearing mice treated with CD22 targeted IL-Doxil (10 mg/kg) was greatly suppressed compared to mice treated with Doxil (10 mg/kg), or nothing.

FIG. 8 illustrates an MTT assay of H1650 cells treated with Doxil or IL-Doxil. Cells were treated for only 1 hr, washed and assayed after 24 hrs. Reported as a % of untreated control. Error bars: standard deviation from 3 experiments.

FIGS. 9A-B illustrate that the prostate cancer cell lines LnCAP (hormone sensitive), PC3 (hormone refractory) and DU145 (hormone refractory) were also found to have significant CD22 expression assessed by flow cytometry. The B-cell lymphoma cell line Ramos was used as a positive control for CD22 staining.

FIG. 10 illustrates CD22 expression in the lung cancer cell line A549 and the prostate cancer cell line DU145 was confirmed at the mRNA level by PCR, using CD22-specific primers. The B-cell lymphoma cell line Ramos was used as a positive control.

FIG. 11 illustrates assaying anti-CD22 antibody for the ability to kill prostate cancer cells in vitro using a complement dependent cytotoxicity (CDC) assay and antibody-dependent cellular cytotoxicity (ADCC). This was performed on the prostate cancer cell lines DU145, LnCaP, and PC3. The antibody alone effectively killed DU145 and PC3 cells but had little effect on LnCaP. PC3 and Du145 represent resistant prostate cancer, which is more difficult to treat and predominates in the human population. When antibody, complement and the effector cells are present, all three prostate cell lines were killed effectively.

### DETAILED DESCRIPTION

1. Introduction

CD22 is a 140 kDa single-pass, transmembrane, sialo-adhesion protein that influences B-cell survival. Nearly all mature B-cells express CD22 as do most non-Hodgkin's lymphoma (NHL). CD22 had been thought to be expressed solely in the cytoplasm and on the surface of B-lymphocytes. The present invention is based, in part, on the discovery of CD22 surface expression on prostate cancer cells and lung cancer cells, particularly non-small cell lung cancer (NSCLC) cells. Expression of CD22 on the surface of NSCLC cells was identified by flow cytometry using a panel of human NSCLC cell lines and by immunohistochemistry (IHC) of several patient samples. Expression was verified by direct nucleic acid sequencing, RT-PCR, immunoblotting and Northern blotting. An anti-CD22 monoclonal antibody (mAb), HB22.7, demonstrated both in vitro and in vivo cytotoxicity in human NSCLC cell lines and xenografts, respectively. Through use of an in vivo orthotopic NSCLC model, it was demonstrated that HB22.7 dramatically inhibited the development of pulmonary metastasis and significantly extended overall survival.

The observation that CD22 is expressed on lung cancer cells and prostate cancer cells reveals a heretofore unexplored mechanism of lung cancer and prostate cancer tumorigenesis, respectively. Moreover, this finding provides a new targeted therapy for lung cancers and prostate cancers, malignancies having few tumor-specific targets.

2. Subjects Who can Benefit from the Present Methods

Patients amenable to treatment or prevention include individuals at risk of lung cancer and/or prostate cancer but not showing symptoms, as well as patients presently showing symptoms. In some embodiments, the subject is exhibiting symptoms of disease and has been diagnosed as having lung cancer and/or prostate cancer. The subject may be in an early stage or late stage of the disease. The subject may or may not have detectable metastasis. In some embodiments, the subject is or appears to be in remission.

In some embodiments, the subject is exhibiting symptoms of lung cancer. For example, the subject may be experiencing or exhibiting dyspnea (shortness of breath), hemoptysis (coughing up blood), chronic coughing or change in regular coughing pattern, wheezing, chest pain or pain in the abdomen, cachexia (weight loss), fatigue, and loss of appetite, dysphonia (hoarse voice), clubbing of the fingernails, dysphagia (difficulty swallowing), predisposition to pneumonia. Subjects may also be experiencing or exhibiting paraneoplastic symptoms, including for example, Lambert-Eaton myasthenic syndrome (muscle weakness due to auto-antibodies), hypercalcemia, syndrome of inappropriate antidiuretic hormone (SIADH), changed sweating patterns, eye muscle problems, and/or muscle weakness in the hands due to invasion of the brachial plexus. Subjects with advanced lung cancer may also experience bone pain.

In some embodiments, the subject is exhibiting symptoms of prostate cancer. For example, the subject may have elevated levels of prostate specific antigen (PSA), e.g., detected in the blood. The subject may also be experiencing or exhibiting frequent urination, nocturia (increased urination at night), difficulty starting and maintaining a steady stream of urine, hematuria (blood in the urine), and dysuria (painful urination), difficulty achieving erection and/or painful ejaculation. Subjects with advanced prostate cancer may be experiencing bone pain, urinary incontinence and/or fecal incontinence.

Generally, the subject does not have and/or has not been diagnosed as having any hematologic malignancy, particularly a hematologic malignancy associated with or mediated by expression or overexpression of CD22. In some embodiments, the subject does not have and/or has not been diagnosed as having any B-cell disorder or disease, including, e.g., any B cell malignancies, autoimmune disease, graft-versus-host disease (GVHD), humoral rejection, and/or post-transplantation lymphoproliferative disorder in an organ transplant recipient. In various embodiments, the subject does not have and/or has not been diagnosed as having a lymphoma (e.g., non-Hodgkin's lymphoma, including Burkitt's lymphoma, Hodgkin's lymphoma, T-cell leukemia lymphoma, or any subtype associated with each), a leukemia (e.g., acute lymphocytic leukemia (ALL), chronic lymphocytic leukemia (CLL), acute myeloid leukemia (AML), chronic myeloid leukemia (CML), adult leukemia), multiple myeloma and/or plasmocytoma.

3. Conditions Subject to Prevention and Treatment

The CD22-antigen binding molecules (antibody and non-antibody) find use in the treatment of lung cancers and prostate cancers that express or overexpress CD22 on their cell surface. The CD22-antigen binding molecules can be administered to a patient to effect the inhibition, reduction, retraction or prevention of proliferation or growth of lung and/or prostate tumors and/or lung and/or cancer cells. In the context of effecting treatment, the patient has a cancer or a tumor burden, and administration of the CD22-antigen binding molecules can reverse, delay or inhibit progression of the disease. In the context of effecting prevention, the patient may be in remission, or may have undergone the removal of a primary tumor, and administration of the CD22-binding molecules can reduce, inhibit or eliminate proliferation and/or growth of metastasis.

Exemplary lung cancers that can be treated or prevented by contacting with the a CD22-antigen binding molecule include without limitation adenocarcinoma, squamous carcinoma, bronchial carcinoma, bronchioalveolar carcinoma, large cell carcinoma, small-cell carcinoma, non-small cell lung carcinoma and metastatic lung cancer refractory to conventional chemotherapy.

Exemplary prostate cancers that can be treated or prevented by contacting with a CD22-antigen binding molecule include without limitation hormone sensitive and hormone refractory prostate cancers. For example, the prostate cancer may be androgen-dependent prostate cancer or androgen-independent prostate cancer. The prostate cancer may be an adenocarcinoma or a small cell carcinoma.

4. Antigen Binding Molecules that Bind to CD22

a. Non-Antibody Antigen Binding Molecules

In various embodiments, the antigen binding molecule is a non-antibody binding protein. Protein molecules have been developed that target and bind to targets in a manner similar to antibodies. Certain of these “antibody mimics” use non-immunoglobulin protein scaffolds as alternative protein frameworks for the variable regions of antibodies.

For example, Ladner et al. (U.S. Pat. No. 5,260,203) describe single polypeptide chain binding molecules with binding specificity similar to that of the aggregated, but molecularly separate, light and heavy chain variable region of antibodies. The single-chain binding molecule contains the antigen binding sites of both the heavy and light variable regions of an antibody connected by a peptide linker and will fold into a structure similar to that of the two peptide antibody. The single-chain binding molecule displays several advantages over conventional antibodies, including, smaller size, greater stability and are more easily modified.

Ku et al. (Proc. Natl. Acad. Sci. U.S.A. 92(14):6552-6556 (1995)) discloses an alternative to antibodies based on cytochrome b562. Ku et al. (1995) generated a library in which two of the loops of cytochrome b562 were randomized and selected for binding against bovine serum albumin. The individual mutants were found to bind selectively with BSA similarly with anti-BSA antibodies.

Lipovsek et al. (U.S. Pat. Nos. 6,818,418 and 7,115,396) discloses an antibody mimic featuring a fibronectin or fibronectin-like protein scaffold and at least one variable loop. Known as Adnectins, these fibronectin-based antibody mimics exhibit many of the same characteristics of natural or engineered antibodies, including high affinity and specificity for any targeted ligand. Any technique for evolving new or improved binding proteins can be used with these antibody mimics.

The structure of these fibronectin-based antibody mimics is similar to the structure of the variable region of the IgG heavy chain. Therefore, these mimics display antigen binding properties similar in nature and affinity to those of native antibodies. Further, these fibronectin-based antibody mimics exhibit certain benefits over antibodies and antibody fragments. For example, these antibody mimics do not rely on disulfide bonds for native fold stability, and are, therefore, stable under conditions which would normally break down antibodies. In addition, since the structure of these fibronectin-based antibody mimics is similar to that of the IgG heavy chain, the process for loop randomization and shuffling can be employed in vitro that is similar to the process of affinity maturation of antibodies in vivo.

Beste et al. (Proc. Natl. Acad. Sci. U.S.A. 96(5): 1898-1903 (1999)) discloses an antibody mimic based on a lipocalin scaffold (Anticalin®). Lipocalins are composed of a β-barrel with four hypervariable loops at the terminus of the protein. Beste (1999), subjected the loops to random mutagenesis and selected for binding with, for example, fluorescein. Three variants exhibited specific binding with fluorescein, with one variant showing binding similar to that of an anti-fluorescein antibody. Further analysis revealed that all of the randomized positions are variable, indicating that Anticalin® would be suitable to be used as an alternative to antibodies. Anticalins® are small, single chain peptides, typically between 160 and 180 residues, which provide several advantages over antibodies, including decreased cost of production, increased stability in storage and decreased immunological reaction.

Hamilton et al. (U.S. Pat. No. 5,770,380) discloses a synthetic antibody mimic using the rigid, non-peptide organic scaffold of calixarene, attached with multiple variable peptide loops used as binding sites. The peptide loops all project from the same side geometrically from the calixarene, with respect to each other. Because of this geometric confirmation, all of the loops are available for binding, increasing the binding affinity to a ligand. However, in comparison to other antibody mimics, the calixarene-based antibody mimic does not consist exclusively of a peptide, and therefore it is less vulnerable to attack by protease enzymes. Neither does the scaffold consist purely of a peptide, DNA or RNA, meaning this antibody mimic is relatively stable in extreme environmental conditions and has a long life span. Further, since the calixarene-based antibody mimic is relatively small, it is less likely to produce an immunogenic response.

Murali et al. (Cell. MoI. Biol. 49(2):209-216 (2003)) discusses a methodology for reducing antibodies into smaller peptidomimetics, they term “antibody like binding peptidomimetics” (ABiP) which can also be useful as an alternative to antibodies.

Silverman et al. (Nat. Biotechnol. (2005), 23: 1556-1561) discloses fusion proteins that are single-chain polypeptides comprising multiple domains termed “avimers.” Developed from human extracellular receptor domains by in vitro exon shuffling and phage display the avimers are a class of binding proteins somewhat similar to antibodies in their affinities and specificities for various target molecules. The resulting multidomain proteins can comprise multiple independent binding domains that can exhibit improved affinity (in some cases sub-nanomolar) and specificity compared with single-epitope binding proteins. Additional details concerning methods of construction and use of avimers are disclosed, for example, in U.S. Patent App. Pub. Nos. 20040175756, 20050048512, 20050053973, 20050089932 and 20050221384.

In addition to non-immunoglobulin protein frameworks, antibody properties have also been mimicked in compounds comprising RNA molecules and unnatural oligomers (e.g., protease inhibitors, benzodiazepines, purine derivatives and beta-turn mimics) all of which are suitable for use with the present invention.

b. Anti-CD22 Antibodies

In various embodiments, the antigen binding molecule is an antibody or antibody fragment that binds to all or any extracytoplasmic domains of CD22. Such anti-CD22 antibodies are useful for treating and preventing lung cancers, prostate cancers and metastasis of lung and prostate cancers.

An antibody suitable for treating and/or preventing lung and/or prostate cancers is specific for at least one portion of an extracellular region of the CD22 polypeptide. For example, one of skill in the art can use peptides derived from an extracellular domain of CD22 to generate appropriate antibodies suitable for use with the invention. Illustrative, non-limiting amino sequences suitable for use in selecting peptides for use as antigens are published as GenBank accession numbers NP_{—}001762.2 (isoform 1); NP_{—}001172028.1 (isoform 2); NP_{—}001172029.1 (isoform 3); and NP_{—}001172030.1 (isoform 4).

A target cell includes any lung cancer cell or prostate cancer cells that expresses or overexpresses CD22. Anti-CD22 antibodies for use in the present methods include without limitation, polyclonal antibodies, monoclonal antibodies, chimeric antibodies, humanized antibodies, human antibodies, and fragments thereof.

The preparation of polyclonal antibodies is well-known to those skilled in the art. See, for example, Green et al., Production of Polyclonal Antisera, in IMMUNOCHEMICAL PROTOCOLS (Manson, ed), pages 1-5 (Humana Press 1992), Coligan et al, Production of Polyclonal Antisera in Rabbits, Rats. Mice and Hamsters, in CURRENT PROTOCOLS IN IMMUNOLOGY, section 241 (1992), which are hereby incorporated by reference.

The preparation of monoclonal antibodies likewise is conventional. See, for example, Kohler & Milstem, Nature 256 495 (1975). Coligan et al., sections 2.5.1-2.6.7, Harlow et al, ANTIBODIES A LABORATORY MANUAL, page 726 (Cold Spring Harbor Pub 1988, and Harlow, USING ANTIBODIES A LABORATORY MANUAL, Cold Spring Harbor Laboratory Press, 1998), which are hereby incorporated by reference Briefly, monoclonal antibodies can be obtained by injecting mice with a composition comprising an antigen, verifying the presence of antibody production by removing a serum sample, removing the spleen to obtain B lymphocytes, fusing the B lymphocytes with myeloma cells to produce hybridomas, cloning the hybridomas selecting positive clones that produce antibodies to the antigen, and isolating the antibodies from the hybridoma cultures. Monoclonal antibodies can be isolated and purified from hybridoma cultures by a variety of well-established techniques. Such isolation techniques include affinity chromatography with Protein-A Sepharose, size-exclusion chromatography, and ion-exchange chromatography. See, e.g., Coligan et al. sections 2.7.1-2.7.12 and sections 2.9.1-2.9.3, Barnes et al., Purification of Immunoglobulin G (IgG), in METHODS IN MOLECULAR BIOLOGY, VOL 10, pages 79-104 (Humana Press 1992).

Methods of in vitro and in vivo multiplication of monoclonal antibodies is well-known to those skilled in the art Multiplication in vitro can be carried out in suitable culture media such as Dulbecco's Modified Eagle Medium or RPMI 1640 medium, optionally replenished by a mammalian serum such as fetal calf serum or trace elements and growth-sustaining supplements such as normal mouse peritoneal exudate cells, spleen cells, bone marrow macrophages. Production in vitro provides relatively pure antibody preparations and allows scale-up to yield large amounts of the desired antibodies. Large scale hybridoma cultivation can be carried out by homogenous suspension culture in an airlift reactor, in a continuous stirrer reactor, or in immobilized or entrapped cell culture. Multiplication in vivo can be carried out by injecting cell clones into mammals histocompatible with the parent cells, e.g., syngeneic mice, to cause growth of antibody-producing tumors. Optionally, the animals are primed with a hydrocarbon, especially oils such as pristane (tetramethylpentadecane) prior to injection. After one to three weeks, the desired monoclonal antibody is recovered from the body fluid of the animal.

Anti-CD22 antibodies can be altered or produced for therapeutic applications. For example, antibodies of the present invention can also be derived from subhuman primate antibody. General techniques for raising therapeutically useful antibodies in baboons can be found, for example, in Goldenberg et al., International Patent Publication WO 91/11465 (1991) and Losman et al., Int. J. Cancer 46:310 (1990), which are hereby incorporated by reference.

Alternatively, therapeutically useful anti-CD22 antibodies can be derived from a “humanized” monoclonal antibody. Humanized monoclonal antibodies are produced by transferring mouse complementarity determining regions from heavy and light variable chains of the mouse immunoglobulin into a human variable domain, and then substituting human residues in the framework regions of the murine counterparts. The use of antibody components derived from humanized monoclonal antibodies obviates potential problems associated with the immunogenicity of murine constant regions. General techniques for cloning murine immunoglobulin variable domains are described, for example, by Orlandi, et al., Proc. Nat'l Acad. Sci. USA 86:3833 (1989), which is hereby incorporated in its entirety by reference. Techniques for producing humanized monoclonal antibodies are described, for example, by Jones et al., Nature 321:522 (1986); Riechmann et al., Nature 332:323 (1988); Verhoeyen et al., Science 239:1534 (1988); Carter et al. Proc. Nat'l Acad. Sci. USA 89:4285 (1992); Sandhu, Crit. Rev. Biotech. 12:437 (1992); and Singer et al., J. Immunol. 150:2844 (1993), which are hereby incorporated by reference.

Anti-CD22 antibodies for use in the present methods also can be derived from human antibody fragments isolated from a combinatorial immunoglobulin library. See, for example, Barbas, et al., METHODS: A COMPANION TO METHODS IN ENZYMOLOGY, VOL. 2, page 119 (1991); Winter et al., Ann. Rev. Immunol. 12:433 (1994), which are hereby incorporated herein by reference. Cloning and expression vectors that are useful for producing a human immunoglobulin phage library can be obtained, for example, from STRATAGENE Cloning Systems (now Agilent Technologies).

In addition, anti-CD22 antibodies for the treatment and/or prevention of lung cancers and/or prostate cancers can be derived from a human monoclonal antibody. Such antibodies are obtained from transgenic mice that have been “engineered” to produce specific human antibodies in response to antigenic challenge. In this technique, elements of the human heavy and light chain loci are introduced into strains of mice derived from embryonic stem cell lines that contain targeted disruptions of the endogenous heavy and light chain loci. The transgenic mice can synthesize human antibodies specific for human antigens, and the mice can be used to produce human antibody-secreting hybridomas. Methods for obtaining human antibodies from transgenic mice are described by Green et al., Nature Genet. 7:13 (1994); Lonberg et al. Nature 368:856 (1994); and Taylor et al., Int. Immunol. 6:579 (1994), which are hereby incorporated by reference.

In various embodiments, the antibodies are human IgG immunoglobulin. As appropriate or desired, the IgG can be of an isotype to promote antibody-dependent cell-mediated cytotoxicity (ADCC) and/or complement-dependent cellular cytotoxicity (CDCC), e.g., human IgG1 or human IgG3.

Antibody fragments for use in the present methods can be prepared by proteolytic hydrolysis of the antibody or by expression in E. coli of DNA encoding the fragment. Antibody fragments can be obtained by pepsin or papain digestion of whole antibodies by conventional methods. For example, antibody fragments can be produced by enzymatic cleavage of antibodies with pepsin to provide a fragment denoted F(ab′)₂- This fragment can be further cleaved using a thiol reducing agent, and optionally a blocking group for the sulfhydryl groups resulting from cleavage of disulfide linkages, to produce Fab′ monovalent fragments. Alternatively, an enzymatic cleavage using pepsin produces two monovalent Fab′ fragments and an Fc fragment directly. These methods are described, for example, by Goldenberg, U.S. Pat. No. 4,036,945 and U.S. Pat. No. 4,331,647, and references contained therein. These patents are hereby incorporated in their entireties by reference. See also, Nisonhoff, et al., Arch. Biochem. Biophys. 89:230 (1960); Porter, Biochem. J. 73:119 (1959): Edelman et al., METHODS IN ENZYMOLOG Y, VOL. 1, page 422 (Academic Press 1967); and Coligan et al. at sections 2.8.1-2.8.10 and 2.10.1-2.10.4.

Other methods of cleaving antibodies, such as separation of heavy chains to form monovalent light-heavy chain fragments, further cleavage of fragments, or other enzymatic, chemical, or genetic techniques can also be used, so long as the fragments bind to the antigen that is recognized by the intact antibody.

For example, Fv fragments comprise an association of VH and VL chains. This association can be noncovalent, as described in Inbar et al., Proc. Nat'l Acad. Sci. USA 69:2659 (1972). Alternatively, the variable chains can be linked by an intermolecular disulfide bond or cross-linked by chemicals such as glutaraldehyde. See. e.g. Sandhu, Crit Rev Biotechnol. 1992; 12(5-6):437-62. In some embodiments, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFv are described, for example, by Whitlow et al, METHODS: A COMPANION TO METHODS IN ENZYMOLOGY, VOL. 2, page 97 (1991); Bird et al, Science 242:423-426 (1988); Ladner, et al, U.S. Pat. No. 4,946,778; Pack, et al, BioTechnology 11:1271 77 (1993); and Sandhu, supra.

Another form of an antibody fragment suitable for use with the methods of the present invention is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick et al, METHODS: A COMPANION TO METHODS IN ENZYMOLOG Y, VOL. 2, page 106 (1991), iv. Small Organic Compounds.

In some embodiments, the anti-CD22 antibody is a single-domain antibody (sdAb) or a nanobody. A single-domain antibody or a nanobody is a fully functional antibody that lacks light chains; they are heavy-chain antibodies containing a single variable domain (VHH) and two constant domains (CH2 and CH3). Like a whole antibody, single domain antibodies or nanobodies are able to bind selectively to a specific antigen. With a molecular weight of only 12-15 kDa, single-domain antibodies are much smaller than common antibodies (150-160 kDa) composed of two heavy protein chains and two light chains, and even smaller than Fab fragments (˜50 kDa, one light chain and half a heavy chain) and single-chain variable fragments (˜25 kDa, two variable domains, one from a light and one from a heavy chain). Nanobodies are more potent and more stable than conventional four-chain antibodies which leads to (1) lower dosage forms, less frequent dosage leading to less side effects; and (2) improved stability leading to a broader choice of administration routes, comprising oral or subcutaneous routes and slow-release formulations in addition to the intravenous route. Slow-release formulation with stable anti-CD22 nanobodies, find use for the treatment and prevention of prostate and lung cancers, avoiding the need of repeated injections and the side effects associated with it. Because of their small size, nanobodies have the ability to cross membranes and penetrate into physiological compartments, tissues and organs not accessible to other, larger polypeptides and proteins.

Numerous antibodies that bind to CD22 are known in the art. Such anti-CD22 antibodies, and fragments thereof, find use for the treatment and prevention of prostate and lung cancers. Illustrative anti-CD22 antibodies for use in the present methods include, e.g., Epratuzumab (humanized LL2) (Furman, et al., Curr Treat Options Oncol. (2004) 5(4):283-8); CAT-8015 (Mussai, et al., Br J Haematol. (2010) 150(3):352-8; inotuzumab ozogamicin (CMC-544) (Wong, et al., Expert Opin Biol Ther. (2010) 10(8):1251-8); RFB4 and BL22 (CAT-3888) (Wayne, et al., Clin Cancer Res. (2010) 16(6):1894-903; and U.S. Pat. Nos. 7,777,019; 7,541,034; and 7,355,012); and HB22.7 (U.S. Patent Publication No. 2007/0264260; O'Donnell, et al., Cancer Immunol Immunother. (2009) 58(10):1715-22). Preferably, the antibodies are humanized for use in treating or preventing lung cancers and/or prostate cancers in humans.

c. Conjugates Comprising an Effector Moiety or a Therapeutic Moiety

In some embodiments, the CD22-antigen binding molecules are administered to the subject or contacted with the lung cancer and/or prostate cancer cell as a conjugate with an effector moiety or a therapeutic moiety. Immunoconjugates comprise the CD22 antigen binding molecules (antibody and non-antibody) conjugated to a cytotoxic agent, such as a chemotherapeutic agent, an anti-neoplastic agent, a cytotoxin, or a radionuclide.

The efficacy of the anti-CD22 antibodies herein can be further enhanced by conjugation to a cytotoxic radionuclide, to allow targeting a radiotherapy specifically to target sites (radioimmunotherapy). Suitable radionuclides include, for example, I¹³¹ and Y⁹⁰, both used in clinical practice. Other suitable radionuclides include, without limitation, In¹¹¹, Cu⁶⁷, Cu⁶⁴, I¹³¹, As²¹¹, Bi²¹², Bi²¹³, and Re¹⁸⁶.

Chemotherapeutic agents useful in the generation of CD22-binding immunoconjugates include, e.g., without limitation, erlotinib, adriamycin, doxorubicin, epirubicin, 5-fluorouracil (5-FU), cytosine arabinoside (“Ara-C”), gemcitabine, cyclophosphamide, thiotepa, busulfan, cyclophosphamide, taxanes, e.g., paclitaxel (Taxol, Bristol-Myers Squibb Oncology, Princeton, N.J.), and docetaxel (Taxotere, Rhone-Poulenc Rorer, Antony, Rnace), methotrexate, pemetrexed, cisplatin, melphalan, vinblastine, bleomycin, etoposide, ifosfamide, mitomycin C, mitoxantrone, vincristine, vinorelbine, carboplatin, teniposide, daunomycin, caminomycin, aminopterin, dactinomycin, mitomycins, esperamicins (see U.S. Pat. No. 4,675,187), 6-thioguanine, 6-mercaptopurine, actinomycin D, VP-16 (etoposide), chlorambucil, melphalan, and other related nitrogen mustards, auristatins including monomethylauristatin E (MMAE), Monomethylauristatin F (MMAF). Other chemotherapeutic agents can find use.

Cytotoxins that find use in the CD22-binding immunoconjugates herein include, for example, diphtheria A chain, Pseudomonas exotoxin A chain, ricin A chain, enomycin, and tricothecenes. Specifically included are antibody-maytansinoid and antibody-calicheamicin conjugates. Immunoconjugates containing maytansinoids are disclosed, for example, in U.S. Pat. Nos. 5,208,020; 5,416,020 and European Patent EP 0 425 235. See also Liu et al., Proc. Natl. Acad Sci. USA 93:8618-8623 (1996). Antibody-calicheamicin conjugates are disclosed, e.g. in U.S. Pat. Nos. 5,712,374; 5,714,586; 5,739,116; 5,767,285; 5,770,701; 5,770,710; 5,773,001; and 5,877,296. Other known cytotoxins, and variants thereof, find use in CD22-binding immunoconjugates.

In some embodiments, the therapeutic agent is an inhibitory nucleic acid. An inhibitory nucleic acid can be delivered to a lung cancer cell or a prostate cancer cell to specifically inhibit expression of a target gene, for example, expression of a gene that mediates the progression of the cancer. Illustrative inhibitory nucleic acids include antisense RNA (asRNA), short inhibitory RNA (siRNA), micro RNA (miRNA) and ribozymes.

In various embodiments, the therapeutic agent is encapsulated in a liposome. Liposomes include emulsions, foams, micelles, insoluble monolayers, liquid crystals, phospholipid dispersions, lamellar layers and the like. In these preparations the composition of the invention to be delivered is incorporated as part of a liposome, alone or in conjunction with a molecule which binds to a desired target, such as antibody, or with other therapeutic or immunogenic compositions. Liposomes for use in the invention are formed from standard vesicle-forming lipids, which generally include neutral and negatively charged phospholipids and a sterol, such as cholesterol. The selection of lipids is generally guided by consideration of, e.g., liposome size, acid lability and stability of the liposomes in the blood stream. A variety of methods are available for preparing liposomes, as described in, e.g., Szoka, et al., Ann. Rev. Biophys. Bioeng. 9:467 (1980), U.S. Pat. Nos. 4,235,871; 4,501,728; 4,837,028 and 5,019,369. The CD22-binding antigen binding molecules (antibody and non-antibody) can be integrated into, attached or conjugated directly to the liposome using methods known in the art. Anti-CD22 antibodies conjugated to liposome-encapsulated doxorubicin has been tested in in vivo animal models. See, e.g., O'Donnell, et al., Invest New Drugs. (2010) 28(3):260-7; O'Donnell, et al., Cancer Immunol Immunother. (2009) 58(12):2051-8 and Tuscano, et al., Clin Cancer Res. (2010) 16(10):2760-8. Those of skill in the art will readily appreciate that the doxorubicin can be exchanged with another therapeutic agent(s) of interest.

In some embodiments, the therapeutic agent is encapsulated in a nanoparticle. Antibody-nanoparticle conjugates are known in the art and described, e.g., in Musacchio, et al., Front Biosci. (2011) 16:1388-412; Cuong, et al., Curr Cancer Drug Targets. (2011) 11(2):147-55; Jain, BMC Med. (2010) 8:83; Sunderland, et al., Drug Development Research (2006) 67(1):70-93; Gu, et al., Nanotoday (2007) 2(3):14-21; Alexis, et al., ChemMedChem. (2008) 3(12):1839-43; Fay, et al., Immunotherapy. (2011) 3(3):381-394; Minko, et al., Methods Mol. Biol. (2010) 624:281-94; and PCT Publ. Nos. WO 2011/046842; WO 2010/040062; WO 2010/047765; and WO 2010/120385, the disclosures of which are hereby incorporated herein by reference in their entirety for all purposes. Known nanoparticle cores find use in encapsulating a therapeutic agent (e.g., a chemotherapeutic agent or an anti-neoplastic agent) for delivering to a lung cancer cell and/or to a prostate cancer cell. A CD22-antigen binding molecule (antibody or non-antibody) can be integrated into, attached or conjugated directly to the nanoparticle core using methods known in the art.

In some embodiments, the encapsulating nanoparticle is a cylindrical PRINT nanoparticle, e.g., as described in Gratton, et al., Proc Natl Acad Sci USA. (2008) 105(33):11613-8. The nanoparticle can be biodegradable or non-biodegradable, as appropriate or desired. Poly(lactic acid-co-glycolic acid) (PLGA), biodegradable poly(L-lactic acid) (PLLA) and PEG-based hydrogels find use as a matrix material in particle drug delivery systems because they are biocompatible, bioabsorbable, and have already shown promise in medical applications. The molecular weight of the polymers and lactic acid to glycolic acid ratios can be easily controlled to tailor release rates and degradation profiles. The PEG hydrogel particles are amenable to the covalent attachment of targeting ligands because of the availability of the amine handle. Using such matrix materials, PRINT particles can be made that contain large quantities of chemotherapy agent, e.g., 5 to 40 wt % of chemotherapy agent (e.g., docetaxel, paclitaxel, cisplatin, gemcitabine, pemetrexed and/or erlotinib).

Conjugates of the antibody and cytotoxic agent can be made using a variety of bifunctional protein coupling agents such as N-succinimidyl-3-(2-pyridyldithiol) propionate (SPDP), iminothiolane (IT), bifunctional derivatives of imidoesters (such as dimethyl adipimidate HCl), active esters (such as disuccinimidyl suberate), aldehydes (such as glutareldehyde), bis-azido compounds (such as bis(p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238:1098 (1987). Carbon-14-labeled 1-isothiocyanatobenzyl-3-methyldiethylene triaminepentaacetic acid (MX-DTPA) is an exemplary chelating agent for conjugation of radionuclide to the antibody. See, WO 94/11026.

Covalent modifications of the anti-CD22 antibodies are also included within the scope of this invention. They may be made by chemical synthesis or by enzymatic or chemical cleavage of the antibody, if applicable. Other types of covalent modifications of the antibody are introduced into the molecule by reacting targeted amino acid residues of the antibody with an organic derivatizing agent that is capable of reacting with selected side chains or the N- or C-terminal residues. A preferred type of covalent modification of the antibodies comprises linking the antibodies to one of a variety of nonproteinaceous polymers, e.g., polyethylene glycol, polypropylene glycol, or polyoxyalkylenes, in the manner well known in the art.

5. Formulation and Administration

a. Formulation

The CD22-binding antigen binding molecules (antibody and non-antibody) can be formulated into pharmaceutical formulations for administration to a patient. Administration of the pharmaceutical formulations can be by a variety of methods. Methods can include systemic administration, wherein the antigen binding molecules are delivered to sites in the body, including the targeted site of pharmaceutical action, via the circulatory system. Systemic administration includes, but is not limited to, oral, intranasal, inhalational, rectal and parenteral (i.e., other than through the alimentary tract, such as intramuscular, intravenous, intra-arterial, transdermal and subcutaneous) administration. In other embodiments administration of the CD22-binding antigen binding molecules is local, e.g., topically or intratumorally.

b. Dosing

The CD22-antigen binding molecules (antibody and non-antibody) can be administered for prophylactic and/or therapeutic treatments. In therapeutic applications, compositions comprising the CD22-binding molecules are administered to a patient suffering from a disease or malignant condition, such as lung cancer or prostate cancer, in an amount sufficient to mitigate, reduce, delay or inhibit the disease and its complications. An amount adequate to accomplish this is defined as a “therapeutically effective dose.” Amounts effective for this use will depend upon the severity of the disease and the general state of the patient's health, and clinical studies are often done to determine the best dose for a given cancer type. An effective amount of the compound is that which provides either subjective relief of a symptom(s) or an objectively identifiable improvement as noted by the clinician or other qualified observer.

In prophylactic applications, compositions containing the CD22-antigen binding molecules are administered to a patient not already in a disease state, or in a state of remission, to prevent the onset of disease. Such an amount is defined to be a “prophylactically effective dose.” In this use, the precise amounts again depend upon the patient's state of health.

Determination of an effective amount is well within the capability of those skilled in the art, especially in light of the detailed disclosure provided herein. Generally, an efficacious or effective amount of CD22-antigen binding molecules is determined by first administering a low dose or small amount of a polypeptide or composition and then incrementally increasing the administered dose or dosages, adding a second or third medication as needed, until a desired effect of is observed in the treated subject with minimal or no toxic side effects. Applicable methods for determining an appropriate dose and dosing schedule for administration of a combination of the present invention are described, for example, in Goodman and Gilman's The Pharmacological Basis of Therapeutics, 11 th Edition, 2006, supra; in a Physicians' Desk Reference (PDR), 64th Edition, 2010; in Remington: The Science and Practice of Pharmacy, 21st Ed., 2006, supra; and in Martindale: The Complete Drug Reference, Sweetman, 2005, London: Pharmaceutical Press., and in Martindale, Martindale: The Extra Pharmacopoeia, 31st Edition., 1996, Amer Pharmaceutical Assn, each of which are hereby incorporated herein by reference.

Exemplary doses of the pharmaceutical formulations described herein, include milligram, microgram or nanogram amounts of the CD22-antigen binding molecules per kilogram of subject or sample weight (e.g., about 0.5 microgram per-kilogram to about 100 micrograms per kilogram, or about 1 microgram per kilogram to about 50 micrograms per kilogram. It is furthermore understood that appropriate doses of the CD22-antigen binding molecules depend upon the potency of the composition with respect to the desired effect to be achieved. When the CD22-antigen binding molecules are to be administered to a mammal, a physician, veterinarian, or researcher may, for example, prescribe a relatively low dose at first, subsequently increasing the dose until an appropriate response is obtained. In addition, it is understood that the specific dose level for any particular mammal subject will depend upon a variety of factors including the activity of the specific composition employed, the age, body weight, general health, gender, and diet of the subject, the time of administration, the route of administration, the rate of excretion, the formulation of the composition, patient response, the severity of the condition, any drug combination, and the and the judgment of the prescribing physician. The dosage can be increased or decreased over time, as required by an individual patient. Usually, a patient initially is given a low dose, which is then increased to an efficacious dosage tolerable to the patient.

The dosage of CD22-antigen binding molecules administered is dependent on the species of mammal, the body weight, age, individual condition, surface area of the area to be treated and on the form of administration. The size of the dose also will be determined by the existence, nature, and extent of any adverse effects that accompany the administration of a particular compound in a particular subject. A unit dosage for administration to a mammal of about 50 to 70 kg may contain between about 10 mg and 2500 mg of the active ingredient, for example, between about 20 mg and 2400 mg active ingredient. Typically, a dosage of the CD22-antigen binding molecules is a dosage that is sufficient to achieve the desired effect.

Optimum dosages, toxicity, and therapeutic efficacy of compositions can further vary depending on the relative potency of individual compositions and can be determined by standard pharmaceutical procedures in cell cultures or experimental animals, for example, by determining the LD50 (the dose lethal to 50% of the population) and the ED50 (the dose therapeutically effective in 50% of the population). The dose ratio between toxic and therapeutic effects is the therapeutic index and can be expressed as the ratio, LD50/ED50. Compositions that exhibit large therapeutic indices are preferred. While compositions that exhibit toxic side effects can be used, care should be taken to design a delivery system that targets such compositions to the site of affected tissue to minimize potential damage to normal cells and, thereby, reduce side effects.

The data obtained from, for example, animal studies (e.g., rodents and monkeys) can be used to formulate a dosage range for use in humans. The dosage of polypeptides of the present invention lies preferably within a range of circulating concentrations that include the ED50 with little or no toxicity. The dosage can vary within this range depending upon the dosage form employed and the route of administration. For any composition for use in the methods of the invention, the therapeutically effective dose can be estimated initially from cell culture assays. A dose can be formulated in animal models to achieve a circulating plasma concentration range that includes the IC50 (the concentration of the test compound that achieves a half-maximal inhibition of symptoms) as determined in cell culture. Such information can be used to more accurately determine a dose range with which to initiate clinical trials in humans. Levels in plasma can be measured, for example, by high performance liquid chromatography (HPLC). In general, the dose equivalent of a polypeptide or composition, is from about 1 ng/kg to 100 mg/kg for a typical subject.

A typical antigen binding molecule composition of the present invention for intravenous administration would be about 0.1 mg/kg to 100 mg/kg per patient per administration. Dosages from 0.1 mg/kg up to about 100 mg/kg per patient per administration may be used. Actual methods for preparing administrable compositions will be known or apparent to those skilled in the art and are described in more detail in such publications as Remington: The Science and Practice of Pharmacy, 21st Ed., 2006, Lippincott Williams & Wilkins.

In one embodiment of the present invention, a pharmaceutical formulation of the present invention is administered, e.g., in a dose in the range from about 1 ng of compound per kg of subject weight (1 ng/kg) to about 100 mg/kg. In another embodiment, the dose is a dose in the range of about 5 mg/kg to about 100 mg/kg. In yet another embodiment, the dose is about 10 mg/kg to about 250 mg/kg. In another embodiment, the dose is about 25 mg/kg to about 150 mg/kg. A preferred dose is about 10 mg/kg.

In various embodiments, the CD22-antigen binding molecules (antibody and non-antibody) are administered via bolus or continuous infusion over a period of time, such as continuous or bolus infusion, once or twice a week. Another route is subcutaneous injection. The dosage depends on the nature, form, and stage of the targeted malignancy, the patients sex, age, condition, prior treatment history, other anti-cancer treatments used (including, e.g. radiation, chemotherapy, immunotherapy, etc.) and other factors typically considered by a skilled physician. For example, lung cancer or prostate cancer patients may receive from about 50 to about 1500 mg/m²/week, specifically from about 100 to about 1000 mg/m²/week, more specifically from about 150 to about 500 mg/m²/week of an anti-CD22 antigen binding molecule, described herein.

Following successful treatment, it may be desirable to have the subject undergo maintenance therapy to prevent the recurrence of the disease or malignant condition treated.

c. Scheduling

Optimal dosing schedules can be calculated from measurements of antigen binding molecules in the body of a subject. In general, dosage is from 1 ng to 1,000 mg per kg of body weight and may be given once or more daily, semiweekly, weekly, biweekly, semimonthly, monthly, bimonthly or yearly, as needed or appropriate. Persons of ordinary skill in the art can easily determine optimum dosages, dosing methodologies and repetition rates. One of skill in the art will be able to determine optimal dosing for administration of a polypeptide or polypeptide composition of the present invention to a human being following established protocols known in the art and the disclosure herein.

The CD22-binding molecules can be administered alone or co-administered in combination with other anti-neoplastic or chemotherapeutic agents. When administered as part of a combination, the CD22-binding molecules can be administered together or separately from the other active agent(s), e.g., as mixtures or in separate formulations. The CD22-antigen binding molecules can be administered via the same or different routes of administration. The CD22-antigen binding molecules can be administered concurrently or sequentially.

Single or multiple administrations of the pharmaceutical formulations may be administered depending on the dosage and frequency as required and tolerated by the patient. In any event, the composition should provide a sufficient quantity of the CD22-antigen binding molecules of this invention to effectively treat the patient. Preferably, the dosage is administered once but may be applied periodically until either a therapeutic result is achieved or until side effects warrant discontinuation of therapy. In some embodiments, the CD22-antigen binding molecules are administered for the remainder of the life of the patient. Generally, the dose is sufficient to treat or ameliorate symptoms or signs of disease without producing unacceptable toxicity to the patient.

The dose can be administered once per week or divided into subdoses and administered in multiple doses, e.g., twice or three times per week. However, as will be appreciated by a skilled artisan, compositions described herein may be administered in different amounts and at different times. The skilled artisan will also appreciate that certain factors may influence the dosage and timing required to effectively treat a subject, including but not limited to the severity of the disease or malignant condition, previous treatments, the general health and/or age of the subject, and other diseases present. Moreover, treatment of a subject with a therapeutically effective amount of a composition can include a single treatment or, preferably, can include a series of treatments.

To achieve the desired therapeutic effect, pharmaceutical formulations may be administered for multiple days at the therapeutically effective daily or weekly dose. Thus, therapeutically effective administration of compositions to treat a disease or malignant condition described herein in a subject may require periodic (e.g., daily or weekly) administration that continues for a period ranging from three days to two weeks or longer. While consecutive daily doses are a preferred route or weekly doses are likely to achieve a therapeutically effective dose, a therapeutically beneficial effect can be achieved even if the compounds or compositions are not administered daily, so long as the administration is repeated frequently enough to maintain a therapeutically effective concentration of the composition in the subject. For example, one can administer a composition every other day, every third day, or, if higher dose ranges are employed and tolerated by the subject, once a week.

In some embodiments, the CD22 antigen binding molecule is administered weekly over the course of 2 to 12 weeks.

d. Combination Therapies

i. Chemotherapy

The CD22-antigen binding molecules can be co-administered with other chemotherapeutic agents as combination therapies. The CD22-antigen binding molecule and the chemotherapeutic agent can be administered together (e.g., as a conjugated moiety or as components of a nanoparticle), or separately. Examples of chemotherapeutic agents that can be co-administered with the CD22-antigen binding molecules include without limitation epidermal growth factor receptor (EGFR), tyrosine kinase inhibitors (erlotinib), folate antimetabolites (pemetrexed), alkylating agents (cisplatin, carboplatin, and oxaliplatin); anti-metabolites (purine or pyrimidine mimetics including for example azathioprine and mercaptopurine); nucleoside analogs (gemcitabine, 5-fluorouracil), plant alkaloids and terpenoids (vinca alkaloids and taxanes); vinca alkaloids (vincristine, vinblastine, vinorelbine, and vindesine); podophyllotoxin (including etoposide and teniposide); taxanes (paclitaxel and docetaxel); topoisomerase inhibitors (Type I inhibitors: camptothecins, irinotecan and topotecan; Type II Inhibitors: amsacrine, etoposide, etoposide phosphate, and teniposide); antineoplastics (dactinomycin, doxorubicin, epirubicin, fludarabine and bleomycin); and Auristatins, including monomethylauristatin E (MMAE), Monomethylauristatin F (MMAF).

Any chemotherapeutic agent being used to treat the cancer of interest can be co-administered in a combination therapy regime with the peptide and polypeptides of the CD22-antigen binding molecules.

ii. Radiation

The CD22-antigen binding molecules can be administered in conjunction with radiological procedures (radiotherapy, radiation therapy). A variety of radiological procedures are available for disease treatments. Any of the procedures know by one of skill can be combined with the polypeptides of the present invention for treatment of a patient. Radiological procedures comprise treatment using radiation therapy to damage cellular DNA. The damage to the cellular DNA can be caused by a photon, electron, proton, neutron, or ion beam directly or indirectly ionizing the atoms which make up the DNA chain. Indirect ionization occurs due to the ionization of water, forming free radicals, notably hydroxyl radicals, which then subsequently damage the DNA. In the most common forms of radiation therapy, the majority of the radiation effect is through free radicals. Due to cellular DNA repair mechanisms, using agents that induce double-strand DNA breaks, such as radiation therapies, has proven to be a very effective technique for cancer therapy. Cancer cells are often undifferentiated and stem cell-like, such cells reproduce more rapidly and have a diminished ability to repair sub-lethal damage compared healthy and more differentiated cells. Further, DNA damage is inherited through cell division, leading to an accumulation of damage to the cancer cells, inducing slower reproduction and often death.

The amount of radiation used in radiation therapy procedure is measured in gray (Gy), and varies depending on the type and stage of cancer being treated and the general state of the patient's health. The dosage range can also be affected by cancer type, for example, the typical curative dosage for a solid epithelial tumor ranges from 60 to 80 Gy, while the dosage for lymphoma ranges from 20 to 40 Gy.

Preventative (adjuvant) doses can also be employed and typically range from 45 to 60 Gy administered in 1.8 to 2.0 Gy fractions (for lung and prostate cancers). Many other factors are well-known and would be considered by those of skill when selecting a dose, including whether the patient is receiving other therapies (for example, but not limited to administration of the CD22-antigen binding molecules, administration of chemotherapies and the like), patient co-morbidities, timing of radiation therapy (for example, whether radiation therapy is being administered before or after surgery), and the degree of success of any surgical procedures.

Delivery parameters of a prescribed radiation dose can be determined during treatment planning by one of skill. Treatment planning can be performed on dedicated computers using specialized treatment planning software. Depending on the radiation delivery method, several angles or sources may be used to sum to the total necessary dose. Generally, a plan is devised that delivers a uniform prescription dose to the tumor and minimizes the dosage to surrounding healthy tissues.

iii. Surgery

The CD22-antigen binding molecules can be administered in conjunction with surgical removal or debulking of tumors. A variety of surgical procedures are available for disease treatments. Any of the procedures know by one of skill can be combined with the polypeptides of the present invention for treatment of a patient. Surgical procedures are the commonly categorized by urgency, type of procedure, body system involved, degree of invasiveness, and special instrumentation.

Examples of surgical procedure can include emergency as well as scheduled procedures. Emergency surgery is surgery that must be done quickly to save life, limb, or functional capacity. Further examples of surgical procedures can include exploratory surgery, therapeutic surgery amputation, replantation, reconstructive, cosmetic, excision, transplantation or removal of an organ or body part, as well as others know in the art. Exploratory surgery can be performed to aid or confirm a diagnosis. Therapeutic surgery treats a previously diagnosed condition. Amputation involves cutting off a body part, usually a limb or digit. Replantation involves reattaching a severed body part. Reconstructive surgery involves reconstruction of an injured, mutilated, or deformed part of the body. Cosmetic surgery can be done to improve the appearance of an otherwise normal structure or for repair of a structure damaged or lost due to disease. Excision is the cutting out of an organ, tissue, or other body part from the patient. Transplant surgery is the replacement of an organ or body part by insertion of another from different human (or animal) into the patient. Removing an organ or body part from a live human or animal for use in transplant is also a type of surgery.

In addition to traditional open surgical procedure that employ large incisions to access the area of interest, surgery procedures further include minimally invasive surgery. Minimally invasive surgery typically involves smaller outer incision(s) which are employed for insertion of miniaturized instruments within a body cavity or structure, as in laparoscopic surgery or angioplasty. Laser surgery involves the use of a laser for cutting tissue instead of a scalpel or similar surgical instruments. Microsurgery involves the use of an operating microscope for the surgeon to see small structures. Robotic surgery makes use of a surgical robot (such as for example the Da Vinci (Intuit Surgical, Sunnyvale, Calif.)), to control the instrumentation under the direction of one of skill, for example, a trained surgeon.

6. Methods of Monitoring

A variety of methods can be employed in determining efficacy of therapeutic and prophylactic treatment with the polypeptides of the present invention. Generally, efficacy is the capacity to produce an effect without significant toxicity. Efficacy indicates that the therapy provides therapeutic or prophylactic effects for a given intervention (examples of interventions can include by are not limited to administration of a pharmaceutical formulation, employment of a medical device, or employment of a surgical procedure). Efficacy can be measured by comparing treated to untreated individuals or by comparing the same individual before and after treatment. Efficacy of a treatment can be determined using a variety of methods, including pharmacological studies, diagnostic studies, predictive studies and prognostic studies. Examples of indicators of efficacy include but are not limited to inhibition of tumor cell proliferation and/or growth and promotion of tumor cell death.

The efficacy of an anti-cancer treatment can be assessed by a variety of methods known in the art. The CD22-antigen binding molecules can be screened for prophylactic or therapeutic efficacy in animal models in comparison with untreated or placebo controls. The CD22-antigen binding molecules identified by such screens can be then analyzed for the capacity to induce tumor cell death or enhanced immune system activation. For example, multiple dilutions of sera can be tested on tumor cell lines in culture and standard methods for examining cell death or inhibition of cellular proliferation and/or growth can be employed. (See, e.g., Maniatis, et al., Molecular Cloning: A Laboratory Manual, Cold Spring Harbor Lab., New York, 1982; Ausubel, et al. Editor, Current Protocols in Molecular Biology, USA, 1984-2008; and Ausubel, et al. Editor, Current Protocols in Molecular Biology, USA, 1984-2008; Bonifacino, et al., Editor, Current Protocols in Cell Biology, USA, 2010; all of which are incorporated herein by reference in their entirety.)

The methods of the present invention provide for detecting inhibition disease in patient suffering from or susceptible to various cancers. A variety of methods can be used to monitor both therapeutic treatment for symptomatic patients and prophylactic treatment for asymptomatic patients.

Monitoring methods entail determining a baseline value of a tumor burden in a patient before administering a dosage of CD22-antigen binding molecules, and comparing this with a value for the tumor burden after treatment, respectively.

With respect to therapies using the CD22-antigen binding molecules, a significant decrease (i.e., greater than the typical margin of experimental error in repeat measurements of the same sample, expressed as one standard deviation from the mean of such measurements) in value of the tumor burden signals a positive treatment outcome (i.e., that administration of the CD22-antigen binding molecules has blocked or inhibited, or reduced progression of tumor proliferation and/or growth and/or metastasis).

In other methods, a control value of tumor burden (e.g., a mean and standard deviation) is determined from a control population of individuals who have undergone treatment with the CD22-antigen binding molecules. Measured values of tumor burden in a patient are compared with the control value (an example of this would be a randomized, placebo controlled clinical trial). If the measured level in a patient is not significantly different (e.g., more than one standard deviation) from the control value, treatment can be discontinued. If the tumor burden level in a patient is significantly above the control value, continued administration of agent is warranted.

In other methods, a patient who is not presently receiving treatment but has undergone a previous course of treatment is monitored for tumor burden to determine whether a resumption of treatment is required. The measured value of tumor burden in the patient can be compared with a value of tumor burden previously achieved in the patient after a previous course of treatment. A significant decrease in tumor burden relative to the previous measurement (i.e., greater than a typical margin of error in repeat measurements of the same sample) is an indication that treatment can be resumed. Alternatively, the value measured in a patient can be compared with a control value (mean plus standard deviation) determined in a population of patients after undergoing a course of treatment. Alternatively, the measured value in a patient can be compared with a control value in populations of prophylactically treated patients who remain free of symptoms of disease, or populations of therapeutically treated patients who show amelioration of disease characteristics. In all of these cases, a significant increase in tumor burden relative to the control level (i.e., more than a standard deviation) is an indicator that treatment should be resumed in a patient.

The tissue sample for analysis is typically blood, plasma, serum, mucous, tissue biopsy, tumor, ascites or cerebrospinal fluid from the patient. The sample can be analyzed for indication of neoplasia. Neoplasia or tumor burden can be detected using any method known in the art, e.g., visual observation of a biopsy by a qualified pathologist, or other visualization techniques, e.g., radiography, positron emission tomography (PET), computerized tomography (CT), ultrasound, magnetic resonance imaging (MRI).

### EXAMPLES

The following examples are offered to illustrate, but not to limit the claimed invention.

### Example 1

### The CD22 Antigen is Broadly Expressed on Lung Cancer Cells

### Materials and Methods

Reagents:

Coomassie Brilliant Blue R, protease inhibitor cocktail tablets and ethylenediaminetetraacetic acid (EDTA) (Sigma Chemical Co., St. Louis, Mo.), goat anti-mouse immunoglobulins fluorescein conjugate (goat anti-mouse Ig-FITC) (Biosource, Camarillo, Calif.), mouse anti-human IgG (H+L) Texas Red conjugate (Rockland Immunochemicals; Gilbertsville, Pa.), anti-mouse HRP antibody (Dako North America, Inc, Carpentaria, Calif.). BCA™ protein assay kit, RPMI 1640 medium, DMEM medium, penicillin-streptomycin and fetal bovine serum (FBS) (Life Technologies, Carlsbad, Calif.), Rituximab (Rituxan) (Genentech (South San Francisco, Calif.). Rabbit anti-CD22 anti-sera (Santa Cruz Biotec). Anti-CD22 used for IHC, NCLCD22-2, (Leice Biosystems, Newcastle UK). The anti-CD22 mAb, HB22.7, was purified from ascites and has been previously characterized (Engel, et al., J Exp Med (1995) 181(4):1581-6). All chemicals were of analytical grade purity.

Cell Lines:

The CD22 positive human Burkitt's B-cell lymphoma line, Ramos (ATCC CRL-1596) and the lung cancer cell lines (A549, H1355, H1975, H460, Calu 1, H1650, H727) were purchased from American Type Culture Collection (Rockville, Md.). The lung cancer cell lines HCC827 and A427 were a kind gift from Dr. Phil Mack (UC Davis Dept. of Internal Medicine), and have been previously characterized (Li, et al., Cancer Res (2010) 70:5942-52; and Huang, et al., Cancer Res (1995) 55:3847-53). All cells were thawed and grown in RPMI-1640 (Ramos) or DMEM (lung cancer lines) supplemented with 10% FBS, 50 units/ml penicillin G, and 50 μg/ml streptomycin sulfate. Cells were maintained in tissue culture flasks at 37° C. in 5% CO₂ and 90% humidity. After two passages, multiple vials were re-frozen and stored in liquid nitrogen for future use. Fresh vials of cells are periodically thawed and used for in vitro experiments to ensure that changes to cells have not occurred over time/passages in culture. For xenograft studies, a fresh vial of A549 or H1650 cells were thawed 7-10 days before tumor cell implantation.

Flow Cytometry (FACS):

FACS was used to assess CD22 surface expression and HB22.7 binding. The primary antibody was added at a 1/50 dilution, then incubated on ice for 45 minutes; cold PBS/FBS (1 ml) was added, microcentrifuged for 5 seconds at 1300×g, then washed again. Goat-anti-mouse-FITC (3 μl) (Biosource, Camarillo, Calif.) was added and incubated on ice for 30 minutes in the dark. Ice-cold PBS/FBS (1 ml) was added and incubated in the dark for 5 minutes. Cells were washed twice then resuspended in 200 μl cold PBS/FBS; then 2% formaldehyde in PBS (300 μl) was added. Cells were then analyzed using a Becton-Dickinson FACSCalibur cytometer with argon-ion laser at 488 nm excitation. A live gate was drawn around intact cells using forward and side scatter to eliminate cell debris; fluorescence in the FL1 (530 nm) detector range was then ascertained. Ten thousand events were collected in the live gate. The mean fluorescent intensity (MFI) was determined for each fluorescent peak using a defined region.

In Vitro Cytotoxicity Assay:

Ramos, A549, or H1650 cells (2-2.5×10⁴ per sample) were plated in triplicate in 96 well round bottom plates in a volume of 100 μL per well. Cells were treated for 1 hour with HB22.7, or rituximab to final concentrations of 25 μg/mL. Control cells received media only. After treatment, plates were washed 3 times in media, then incubated at 37° C. in 5% CO₂ and 90% humidity for 5 days. Viability was assessed by trypan blue exclusion; experiments were done in triplicate and results were expressed as % of control (untreated cells) with the error bars representing the standard deviation.

Immunoblotting, Northern Blotting, and RT-PCR:

CD22 Immunoblotting was done as previously described (Tuscano, et al., Blood (1999) 94(4):1382-92; Tuscano, et al., Eur J Immunol (1996) 26(6):1246-52). Briefly indicated cells were lysed in 125 μL of Cell Lytic M lysis buffer supplemented with a protease inhibitor cocktail tablet, sodium orthovanadate, and 2-glycerophosphate. Cells were lysed on ice for 30 minutes with occasionally vortexing. Lysates (50 μg protein per lane) were run on a 10% SDS-PAGE gel, followed by transfer to a nitrocellulose membrane. Membranes were blocked with 5% BSA in Tris-buffered saline with Tween-20 (TBS-T), rinsed, then incubated at 4° C. overnight in primary antibodies (anti-CD22) diluted 1:1000 in 5% BSA in TBS-T. Washed membranes were incubated for 1 hour at room temperature with anti-mouse HRP conjugate diluted 1:10,000 in 5% BSA in TBS-T. Membranes were washed 4 times in TBS-T, then probed with Advanced ECL detection reagent.

RT-PCR was done as described previously (Tuscano, et al., Blood (1999) 94(4):1382-92). Briefly, semi-quantitative PCR was done by extracting total RNA from cells using the RNeasy mini kit (Qiagen, Valencia, Calif.) following the manufacturer's instructions. cDNA was synthesized from 500 ng of total RNA using the SuperScript® III First-Strand Synthesis System for RT-PCR (Invitrogen). PCR conditions, including CD22 primer selection, concentration and annealing temperature, were previously optimized. GAPDH was used as a reference gene.

CD22 Northern blot analysis was done as described (Wilson, et al., J Exp Med (1991) 173(1): 137-146). Briefly, total was size separated via agarose-formaldehyde PAGE and transferred to a nylon membrane. CD22-specific RNA was detected with a DIG-dUTP-labeled DNA probe generated using CD22-specific PCR primers and following the manufacturer's recommendations (Roche).

Internalization Assay:

The internalization assay was purchased from Advanced Targeting Systems (San Diego, Calif.) and done as described in the package insert. Briefly, saporin-conjugated goat anti-human IgG (Hum-ZAP) and goat IgG isotype control (Goat IgG-SAP) secondary antibodies were provided and have been previously described (Kohls and Lappi (2000)BioTechniques 28(1):162-165. A549 and H727 cells were added to 96-well microplates in 90 μL media and allowed to adhere overnight. 2×10⁴ Ramos cells were plated in 90 μL media the day of the experiment. Serial dilutions of HB22.7 were incubated with 10 μg/mL of the secondary conjugate, Hum-ZAP, for 15 minutes at room temperature. 10 μL of the HB22.7-Hum-ZAP conjugate was added to each well giving a final concentration of 1 μg/mL Hum-ZAP. Goat IgG-SAP was used as a non-targeted saporin control for Hum-ZAP. After 72 hours of incubation, cell viability was assessed using the CellTiter 96 AQueous One Solution Cell Proliferation Assay according to the manufacturer's instructions. The plates were read at 490 nm in a microplate reader. Percent cell viability is defined by the following equation: (OD of treatment/OD of control)*100, where the control is represented by cells treated with media alone and has been previously shown to correlate with the degree of internalization.

ADCC/CDC Assay:

For the ADCC assay, PBMCs were cells were isolated from whole blood collected into citrated vacuum tubes from healthy volunteers using standard protocol. Following isolation, the PBMCs were placed into culture in RPMI plus 10% FCS. Cells were activated using Human IL-2 (Proleukin, Chiron Inc.) at 1000 units/ml overnight at 37° C. The activated PBMCs were co-incubated with the A549 lung cancer cells which were plated in a 96-well plate in triplicates 12-24 hours before the assay. As a source of complement, human serum was added at a 1/10 dilution. The activated PBMC are added at 10× the number of target cells. The cells were incubated for two days at 37° C. and 5% CO₂ in a humidified chamber. The number of live cells was determined by trypan blue exclusion using a microscope at high power (40×) and averaging 10 fields. Human serum complement was purchased from Quidel, San Diego, Calif., and stored frozen at −80° C. in aliquots until used, at which time the aliquots were rapidly thawed and used at various dilutions in the media for the CDC assay.

Xenograft Studies:

Female, 6-8 week old Balb/c nude mice were obtained from Harlan Sprague Dawley (Indianapolis, Ind.) and maintained in micro-isolation cages under pathogen-free conditions in the UC Davis animal facility. Three days after whole body irradiation (400 rads), 1×10⁶ A549, PC-3, or H1650 cells were implanted subcutaneously on the left flank. Either one day after tumor implantation (preemptive), or once approximately 100 mm³ tumors have been established (˜14d) mice were randomly divided into treatment groups (n=8-10 per group): 1.4 mg of HB22.7, Rituximab, or IgG (control). Mice were administered treatment on days 1, 7, 14, and 21 after tumor implantation (preemptive) or weekly for four weeks after tumor establishment. All treatments were administered via the tail vein. Tumors were measured twice per week using a caliper, and tumor volumes were calculated using the equation: (length×width×depth)×0.52. Mice were euthanized when the tumor reached 15 mm in any dimension, if they became moribund, or at the end of the 84 day study.

The orthotopic xenograft model has been previously described (Hatakeyama, Methods in Enzymology. (2010) 479:397-411; and Guilbaud, et al., Anti-Cancer Drugs, (1997) 8(3):276-82). Briefly 10⁶ A549 cells were resuspended in 100 μl of media and injected via tail vein. Animals were treated with HB22.7 (1.4 mg) of Ig control on days 1, 7, 14, and 21. Animals were euthanized when they became moribund (although the majority of the untreated animals died by day 14) and lungs were harvested and examined histologically. To facilitate comparison of the development of lung metastasis, a cohort of treated animals were also euthanized at day 14. To facilitate a comparison of overall survival, a cohort of treated animals (5) were not euthanized and monitored for an additional 60 days.

Mice were assessed for toxicity by twice-weekly measurement of their weight, activity, and blood counts for the first 28 days, then weekly for the rest of the 84-day study period (standard assessment of toxicity by the UC Davis School of Veterinary Medicine Lab Animal Clinic).

I-PET:

Copper-64 labeled HB22.7 was used to determine the ability of HB22.7 to specifically target A549 in vivo done as previously described (Martin, et al., Mol Imaging Biol. (2009) 11(2):79-87). ⁶⁴Cu (a positron emitter) combines all three modes of decay: electron capture (41%), beta − (40%) and beta + (19%) making it a useful radionuclide for both imaging and therapy. ⁶⁴Cu was produced on the biomedical cyclotron at Washington University and supplied as ⁶⁴CuCl₂ (0.1M HCl). The bifunctional chelating agent, DOTA (1,4,7,10-tetraazacyclododecane N,N′,N″,N′″-tetraacetic acid (DOTA) contains a reactive functionality to form a covalent attachment to proteins and a strong metal-binding group to chelate radiometals. DOTA-HB22.7 was prepared by incubation with DOTA-NHS-ester at pH 5.5. DOTA-HB22.7 was labeled with ⁶⁴Cu-acetate in 0.1M ammonium acetate, pH 5.5. After incubation 1 mM EDTA terminated the reaction. HPLC purification was then performed to purify the ⁶⁴Cu-DOTA-HB22.7.

Statistical Analysis:

In vitro cytotoxicity data and apoptosis data was analyzed by a two-tailed, unpaired Student's t-test. Tumor volume data was analyzed using Kaplan-Meier curves. For this analysis, an “event” was defined as tumor volume reaching 400 mm3 or greater. Each individual mouse was ranked as a 1 (event occurred) or a 0 (event did not occur) and the time to event (in days) was determined. When an individual was ranked as 0 (event did not occur), a time to event of 88 days (number of days in the 12.5 week study) was recorded. Chi-squared and p values were determined by the Log-rank test. All statistical analysis was performed using GraphPad Prism software (San Diego, Calif.). A p value of <0.05 was considered significant.

### Results

Expression of CD22 in Lung Cancer.

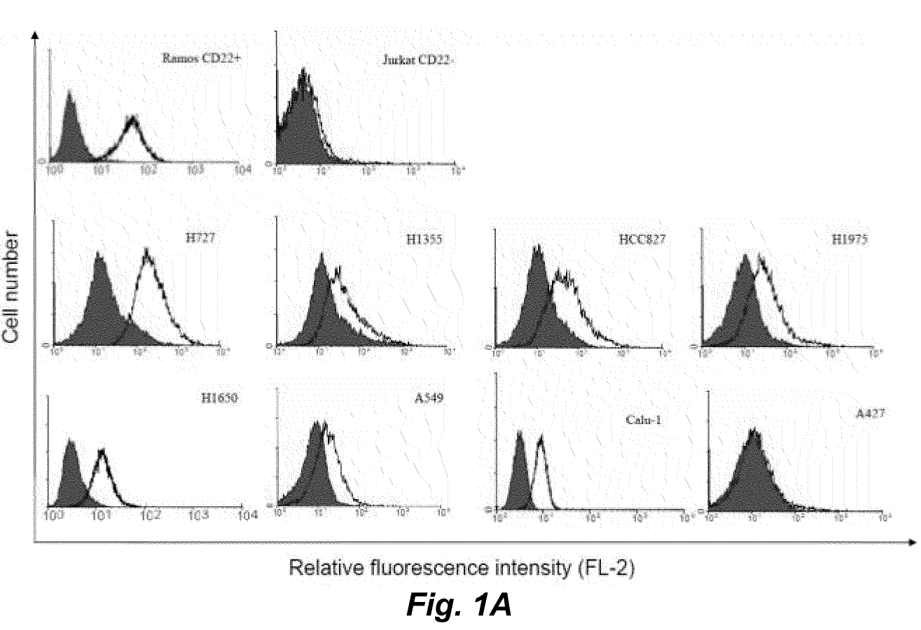

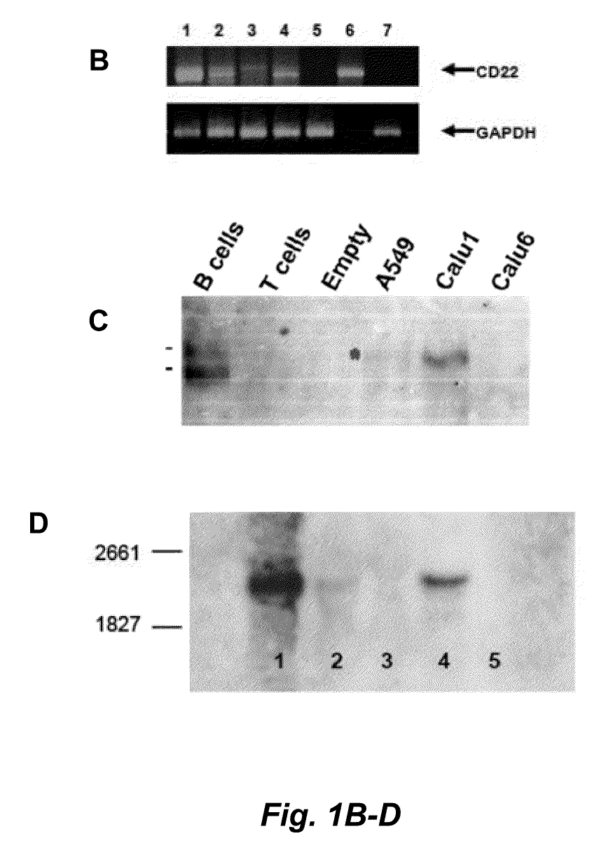

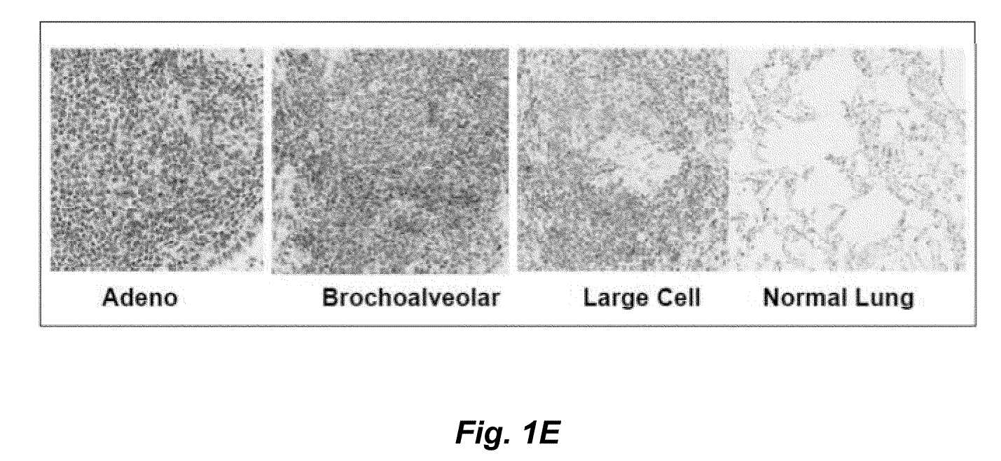

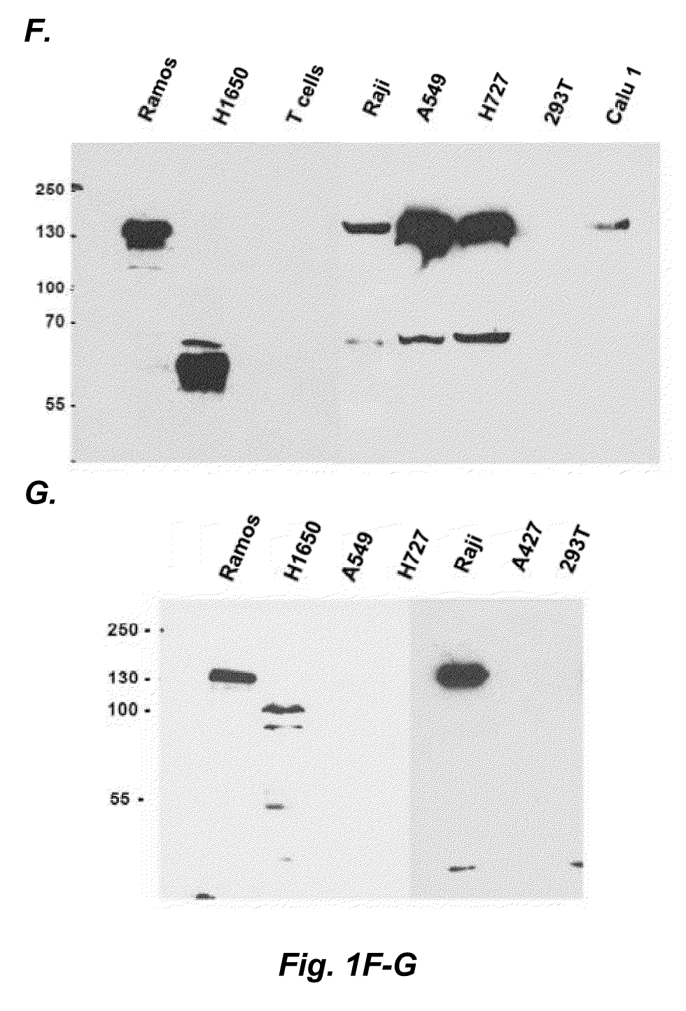

The HB22.7 anti-CD22 mAb recognized an epitope on the surface of A549 NSCLC cells. This finding prompted us to examine CD22 expression by flow cytometry in a panel of NSCLC cell lines representing the major lung cancer subtypes: adenocarcinoma (A549, H1355, H1975, HC827, H460), squamous cell (Calu 1), bronchioalveolar (BAC) (H1650), epidermoid (A427), and carcinoid (H727). HB22.7 bound all of the cell lines except A427 and HC827, in some cases at levels nearly as high (e.g. H727) as on Ramos B-cell NHL cells, (FIG. 1A). The surface expression was consistent using other anti-CD22 mAbs as well, including HB22.2712 and HD617.

To verify that CD22 was expressed in the NSCLC lines that were positive by flow cytometry, mRNA was isolated from selected NSCLC cell lines and normal bronchial epithelial cells (BEC) and quantitative reverse transcriptase polymerase chain reactions (RT-PCR) was done using human CD22-specific oligonucleotides, (FIG. 1B). A cDNA fragment of the predicted length was amplified from Ramos B-cells, A549, H727, H1650 and CD22-containing plasmid but not from mRNA isolated from BEC and A427 cells consistent with the flow cytometry data. Next, an anti-CD22 immunoblot (IB) analysis was performed to see if a protein band within the expected molecular weight range for human CD22 could be detected in protein lysates from flow cytometry-positive NSCLC (but not in lysates from Jurkat T-cells, the negative control), (FIG. 1C). Clear bands in the appropriate size range were detected in the primary B-cell (positive control) but not in the primary T-cell (negative control). Bands in a similar size range were detected in lanes for CD22 PCR-positive cell lines Calu1 and A549, but not in the sample lane for the anti-CD22-flow cytometry negative NSCLC line, Calu6. To verify expression and transcript size, a Northern blot was done with total RNA from Ramos B-cells, A549, H1650, H727, and A427, (FIG. 1D). This revealed clear mRNA expression in A549 and H727 cells, low level expression in H1650 and no detected expression in A427.

To determine if the CD22 sequence was the same as that found in B-cells, all 2541 base pairs of CD22 cDNA from A549 cells were sequenced; the sequence in A549 was identical to the published sequence of CD22 isolated from B-cells (Wilson, et al., J Exp Med (1991) 173(1): 137-146). To demonstrate that CD22 was not aberrantly expressed in these NSCLC cell lines, archived tissue blocks from patients with NSCLC and normal lung tissue were obtained and immunoperoxidase (IP) staining for CD22 expression was performed on paraffin embedded sectioned material. Because CD22 is heavily post-translationally modified (siaylation) (Crocker, et al., Nat Rev Immunol (2007) 7(4):255-66; Shan, et al., J Immunol (1995) 154(9):4466-75). CD22 has been notoriously difficult to detect via IHC, however, using anti-CD22 mAb conjugated with peroxidase a significant degree of CD22 staining was detected in three different NSCLC tumor types, (FIG. 1E). The anti-CD22 IP signal was often intense in part of the tumors, but was weak or undetectable in the surrounding normal lung tissue. Several additional NSCLC patient specimens also stained CD22-positive by IHC but a cytoprep of normal human lung cells obtained from bronchoscopy was CD22 negative.

CD22-Mediated Lung Cancer Cell Killing and Receptor Internalization:

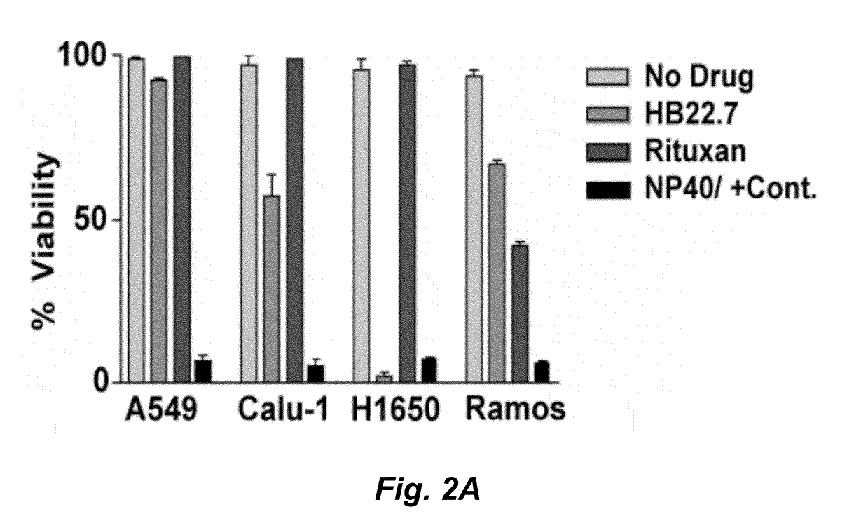

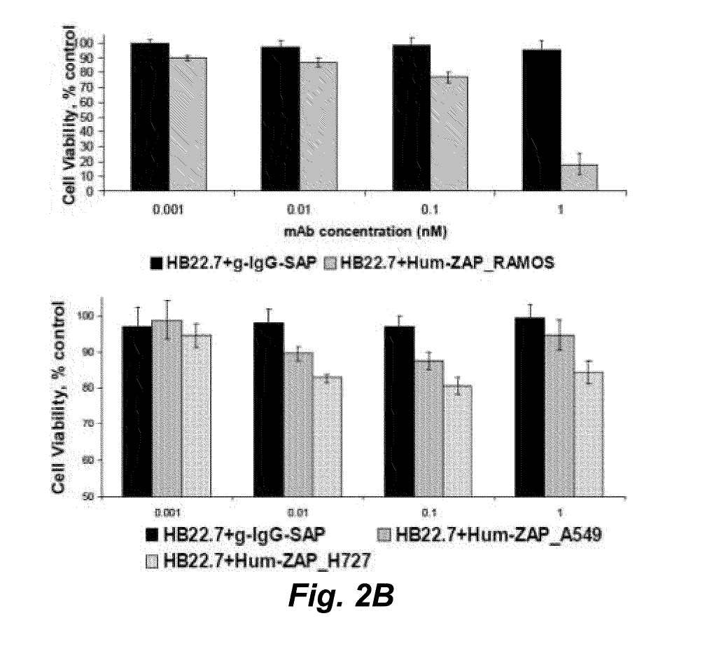

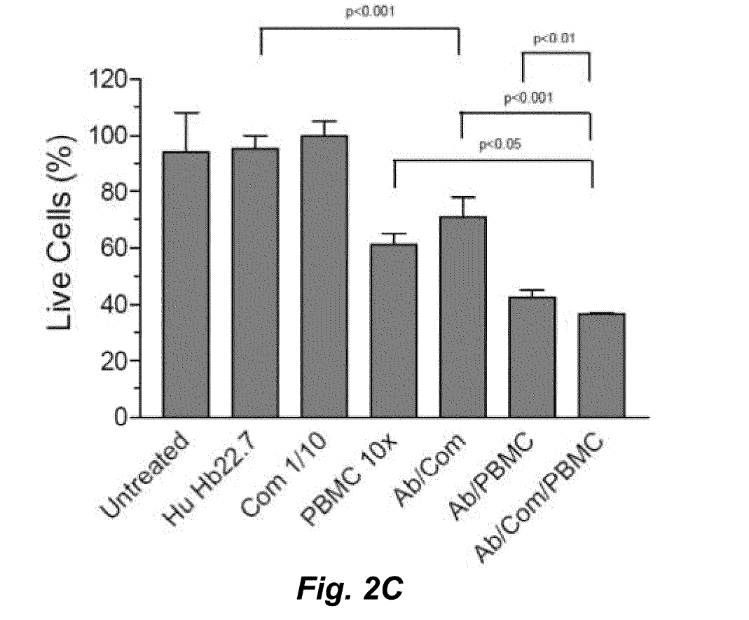

Crosslinking of CD22 with ligand-blocking anti-CD22 mAbs induces cell death in B-cell NHL (Tuscano, et al., Blood (1999) 94(4):1382-92; and Tuscano, et al., Blood (2003) 101(9):3641-7), therefore it was assessed whether this was true for CD22-expressing NSCLC as well. Three NSCLC lines, as well as Ramos NHL cells, were tested for their responsiveness to HB22.7-mediated CD22 crosslinking. As expected, after 48 hours the cytotoxic effects of HB22.7 and rituximab (anti-CD20 control mAb) were observed in Ramos cells. However, the viability of H1650 and Calu-1 was greatly decreased by treatment with HB22.7; as expected, no cytotoxic response was induced in the NSCLC lines treated with rituximab (FIG. 2A).

While mAb-bound CD22 is known to mediate CD22 internalization in B-cells (Engel et al., J Exp Med (1995) 181(4):1581-6), in NSCLC the issue of receptor internalization has never been explored. CD22 internalization was examined in A549 and H727 NSCLC cell lines that have intermediate and high levels of CD22 surface expression and then compared to Ramos B-cells (FIG. 2B). Using a novel toxin-conjugated method with the degree of internalization being proportional to the degree of cytotoxicity, this study confirmed that HB22.7-mediates over 80% of CD22 internalization in B-NHL cells and reveals that there is 20% and 10% internalization in A549 and H727 cell respectively. The difference in magnitude may be due to different CD22 expression levels in NHL versus NSCLC cells. In primary B cells and B NHL cells, CD22 is internalized after being bound by ligands (Crocker, et al., Nat Rev Immunol (2007) 7(4):255-66; Shan, et al., J Immunol (1995) 154(9):4466-75).

It is unknown if anti-CD22-mediated tumor suppression is mediated through host immune effector mechanisms such as antibody-dependent-cellular-cytotoxicity (ADCC) or complement-dependent-cytotoxicity (CDC) or direct cytotoxic effects. Recruitment of antibody-mediated host immune effector mechanisms can be altered by receptor internalization (Weiner and Carter, Nature (2005) 23(5):556-7). The possibility that humanized HB22.7 (hHB22.7) could mediate ADCC and/or CDC in NSCLC cells was investigated using A549 cells (FIG. 2C). Additional A549-killing occurred when complement was added to huHB22.7 (approximately 2% killing by complement alone; approximately 40% killing with complement plus huHB22.7). Little effect resulted from the addition of peripheral blood mononuclear cells (PBMC) to huHB22.7 above that seen with PBMCs alone, (FIG. 2C).

HB22.7 Effectively Targets NSCLC/A549 Xenografts In Vivo:

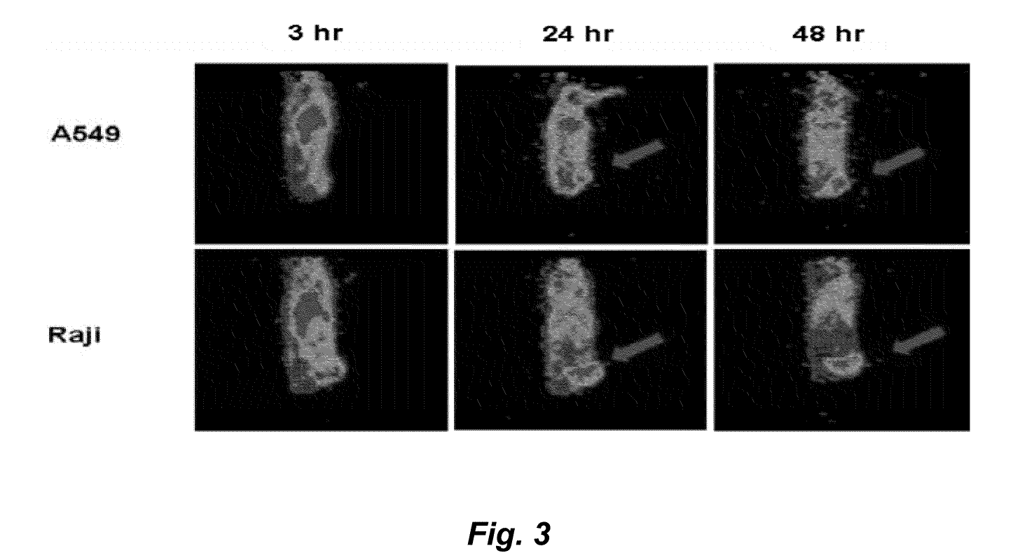

It was previously demonstrated that in vivo targeting of NHL xenografts could be effectively monitored with immuno positron emission tomography (i-PET) using 64Cu-DOTA-HB22.722. Using the same anti-CD22 i-PET methodology, it was shown that HB22.7's biodistribution and specific targeting to NSCLC xenografts was similar to that seen for HB22.7 treated Raji-NHL xenografts, (FIG. 3). The majority of the immunoconjugate is cleared from the blood pool at 48 hours and at this time point there is specific NSCLC uptake and very little uptake in other organs including normal lung.

Activity of Anti-CD22-Blocking mAb HB22.7 Against NSCLC In Vivo:

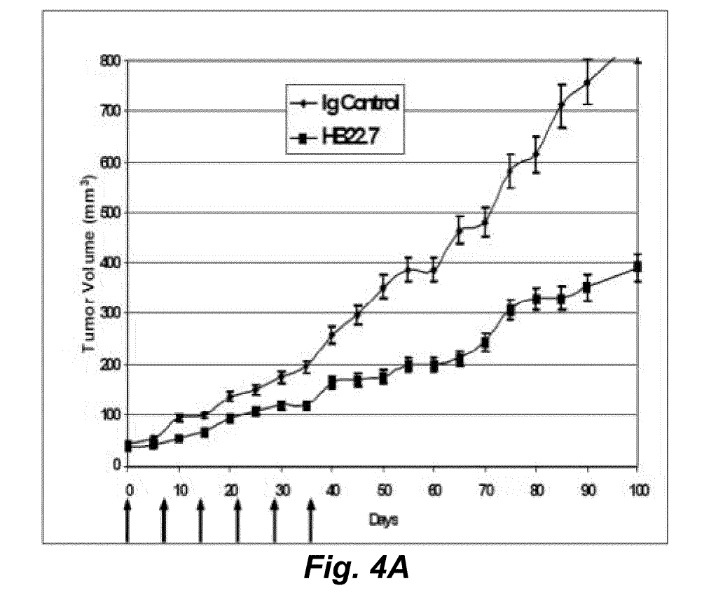

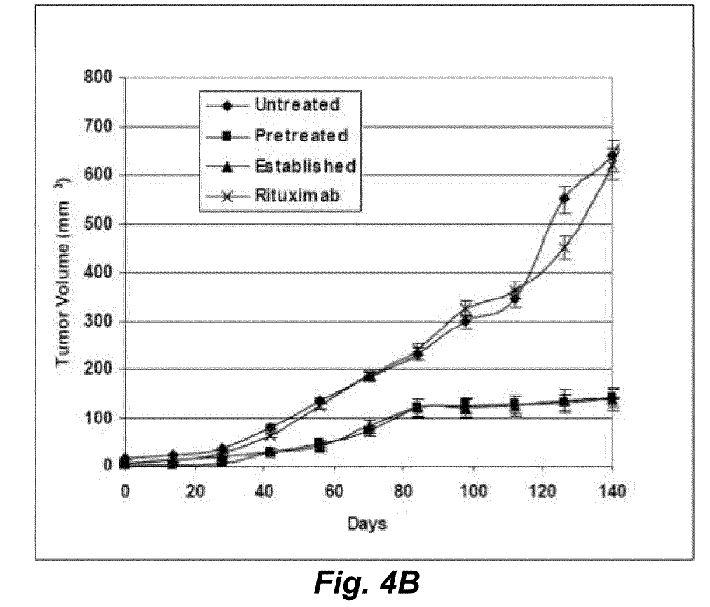

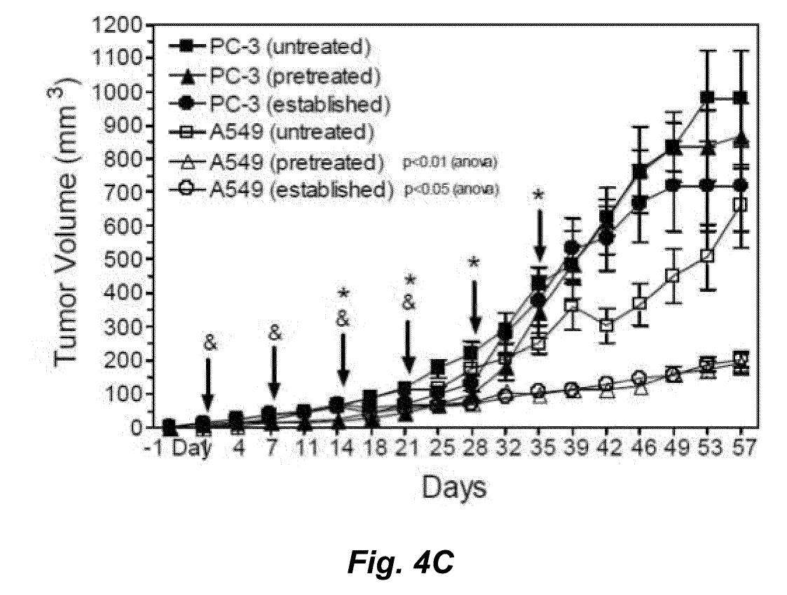

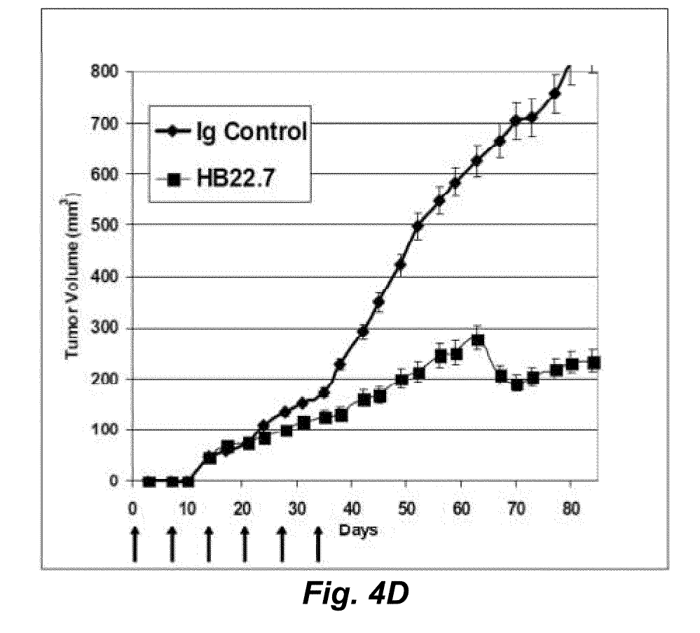

Based on our discovery that CD22 is expressed on NSCLC cells, and that HB22.7 bound to a majority of the NSCLC cell lines, xenograft trials were initiated to assess the pre-clinical efficacy of HB22.7, (FIGS. 4A-D). As H1650 (BAC) cells are sensitive to the cytotoxic effects of HB22.7 in vitro, the ability of HB22.7 to inhibit H1650 tumor growth in nude mice was tested (FIG. 4A). This shows that HB22.7 had significant efficacy against H1650 tumors in nude mice with greater than a 50% reduction in tumor volume at the end of the study (p<0.001).

Since A549 NSCLC cells were resistant to HB22.7-induced cytotoxicity in vitro, the pre-clinical efficacy of HB22.7 was assessed in a CD22 positive, but resistant cell line (FIG. 4B). HB22.7 did significantly retard tumor growth in A549-bearing mice, as compared to the untreated, and anti-CD20 (rituximab) control (p<0.05). The tumor specificity of this effect was demonstrated in a repeat study with mice bearing either A459 or PC-3 (prostate cancer) xenografts, (FIG. 4C). HB22.7 did not significantly retard growth of the PC-3 xenografts but again demonstrated consistent reduction in growth of A549 cells. The efficacy against A549 xenografts was surprising considering that HB22.7 had little in vitro cytotoxicity against A549 cells. Nevertheless, four independent trials verified the efficacy of HB22.7 against human NSCLC xenografts. Why HB22.7 demonstrated in vivo efficacy against A549 xenografts that exhibited resistance to HB22.7 in vitro is not clear, but previous in vitro studies demonstrated that host immune effector mechanisms may be contributing (FIG. 2C). Pre-irradiation of xenografted mice increases the xenograft take-rate by suppressing residual immunity. Nude mice have residual NK cell activity capable of ADCC as well as complement. Because HB22.7 has little cytotoxic activity in A549 cells in vitro but demonstrates activity in xenograft models, this residual immune function may contribute to HB22.7-mediated efficacy. An A549 xenograft trial without radiation before tumor-implantation was done to study HB22.7's mechanism of action (FIG. 4D). This trial verified the activity of HB22.7 against NSCLC A549 xenografts and that activity seemed enhanced suggesting that ADCC and/or CDC contribute.

An Orthotopic Model of Lung Cancer Metastasis:

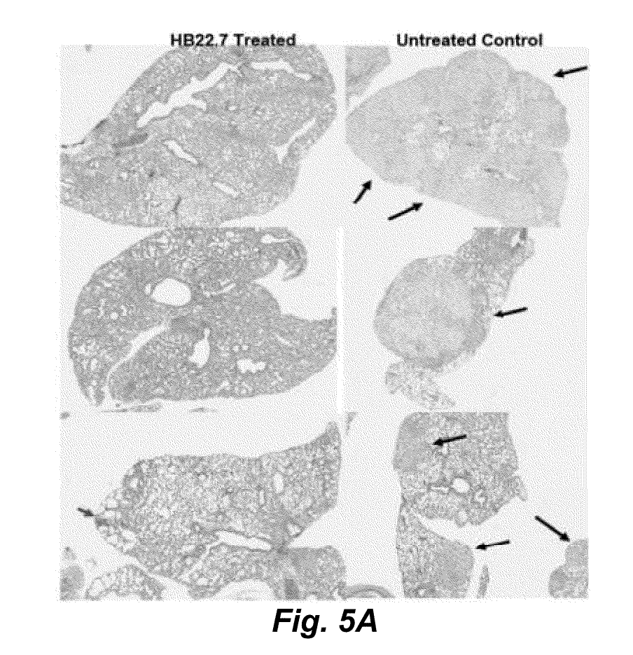

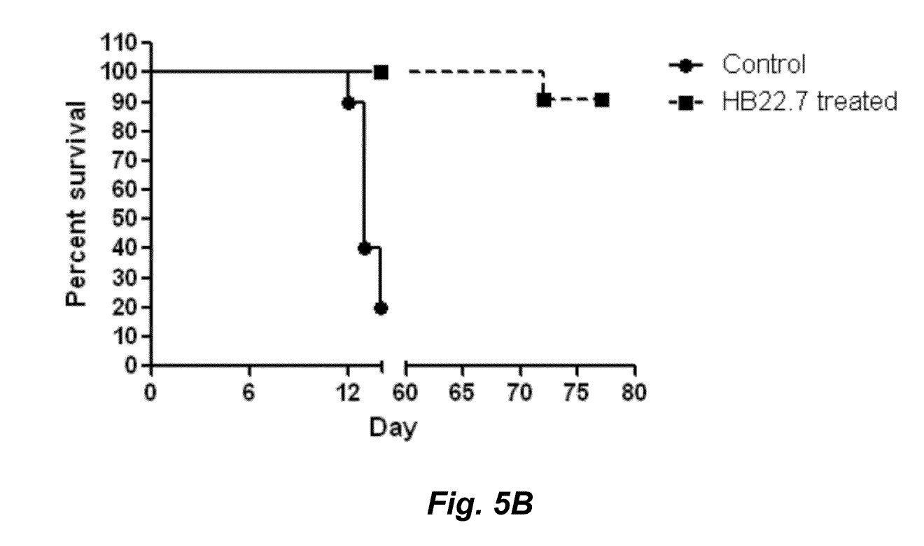

CD22 mediates B-cell homing to endothelial cells 23,24. To confirm that CD22 promotes or facilitates lung cancer metastasis, the anti-CD22 mAb HB22.7 was used in an orthotopic model of lung cancer, seeking to prevent lung metastasis after intravenous injection of NSCLC cells. Forty days after A549 tumor injection with or without HB22.7 mice were euthanized, lungs harvested and examined histologically for metastases (FIG. 5A). The differences were dramatic—most of the lungs from the untreated mice had a heavy tumor burden and one was nearly replaced with tumor (upper right black arrows); in HB22.7-treated mice the lungs were virtually devoid of tumor with the exception of one micro-metastasis (red arrow, lower left). This demonstrates that CD22 plays a significant effect on the development of lung cancer metastasis. This model was also used to assess the effects of HB22.7 treatment on survival. Animals that were not euthanized were either continued on weekly injections of HB22.7 or observed. This demonstrated a significant improvement in survival with over 90% of treated mice alive at the end of the 84 day trial; 100% of the untreated mice were dead by day 14 (p<0.0001) (FIG. 5B).

### Discussion

The analysis of the effects of HB22.7 on NHL uncovered an unexpected finding—the anti-CD22 mAb recognized an epitope on the surface of A549 NSCLC cells. This finding prompted examination of CD22 expression by flow cytometry in a panel of NSCLC cell lines representing the major NSCLC subtypes: adenocarcinoma (A549, H1355, H1975, HC827, H460), squamous cell (Calu 1), bronchioalveolar (BAC) (H1650), epidermoid (A427), and carcinoid (H727). HB22.7 bound all of the cell lines except A427 and HC827, in some cases at levels nearly as high (e.g. H727) as on Ramos B-cell NHL cells (FIG. 1). All available evidence presented herein and elsewhere (Martin, et al., Mol Imaging Biol. (2009) 11(2):79-87; Wilson, et al., J Exp Med (1991) 173(1):137-46; and Postema, et al., Clin Cancer Res (2003) 9(10 Pt 2):39955-40025) suggests that CD22 is not expressed in normal lung epithelial cells and CD22 expression is a common but distinct feature of malignant lung cells. Scrutiny of publicly available cDNA microarray databases (NCBI GEO) indicates that CD22 is expressed in several lung cancer cell lines that have not been tested (some at relatively high levels, e.g. H1770, EBC-1, and LU65); this provides independent verification of our findings. This was verified using RT-PCR, I-PET, Northern blot, and IHC of patient tumor specimens (FIGS. 1&3). The pattern of CD22 expression assessed via IHC was patchy yet distinct, with expression appearing more prominent in tightly packed clusters of tumor cells (FIG. 1). This can be a manifestation of the adhesive properties of CD22 which, in part, contributes to CD22-mediated lung cancer metastasis. The transcript size and sequence of CD22 found in NSCLC is identical to that in B-cells and thus the surface expression pattern is the only anomaly that likely mediates a selective advantage in terms of metastasis and possibly growth. The specific role of CD22 in B-cells remains controversial, but most agree that it mediated adhesion and modulates B-cell receptor-mediated signals (Tedder, et al., Annu Rev Immunol (1997) 15:481-504; Tedder, et al., Adv Immunol (2005) 88:1-50). There is also evidence that the CD22 ligand binding domain mediates a specific survival signal in normal as well as malignant B-cells (Haas, et al., J Immunol (2006) 177(5):3063-73). It has been demonstrated that the CD22 ligand blocking mAb HB22.7 has significant lymphomacidal properties; however its potency is variable.

While the mechanism remains poorly understood, previous studies using murine models have suggested that the effects of HB22.7 are specifically mediated by inhibiting the effects of CD22 ligand binding. Selective killing of NSCLC cells in vitro and in vivo (FIG. 4) has also been demonstrated. While the growth of both A549 and H1650 xenografts were inhibited by HB22.7, only H1650 demonstrated in vitro cytotoxicity, and thus the possibility that host immune effector mechanisms are contributing was examined (FIG. 2). An ADCC and CDC assay demonstrated that complement plays a role in the in vivo activity, a system which remains intact in nude mouse xenograft models. Previous studies with mAb that bind internalizing receptors have suggested that host immune effector mechanism has less of a role. While CD22 on A549 and H727 NSCLC cells did demonstrate some internalization, it was approximately 50% less than what was observed on Ramos B cells (FIG. 2). CD22 has been shown to mediate both homotypic and heterotypic adhesion with known CD22 ligand bearing cells that include nearly all hematopoietic cell types as well as endothelial cells (Engel, et al., J Exp Med (1995) 181(4):1581-6; Wilson, et al., J Exp Med (1991) 173(1): 137-146; and Crocker, et al., Nat Rev Immunol (2007) 7(4):255-66). Because CD22 is aberrantly expressed on lung cancer cells, and has a role in homing, it was determined whether CD22 mediates lung cancer metastasis in lung cancer cells. To test this, an intravenous orthotopic model of lung cancer metastasis was used (Hatakeyama, et al., Methods in Enzymology. (2010) 479; 397-411). Treatment with HB22.7 resulted in an enormous reduction in the number and size of tumor implanted in the lung. There were virtually no tumors detected in the lung of animals treated with HB22.7 (FIG. 5). This model also provided an opportunity to study the effects of treatment with HB22.7 on survival. While some animals were euthanized for histologic examination, those that were observed demonstrated a significant improvement in overall survival when treated with HB22.7 with 100% of untreated animals having succumbed by day 14 and over 90% of the treated animals being alive at day 60 (FIG. 5).

In summary, the identification of CD22 on lung cancer represents a milestone for development lung cancer specific therapeutics. In addition, examination of the role of CD22 provides for a better understanding of the pathogenesis and invasiveness of lung cancer.

### Example 2

### Liposome-Encapsulated Chemotherapeutic Agents Coated with Anti-CD22 Antibodies Inhibit Lung Cancer Growth

Doxorubicin-Carrying, HB22-7-Coated IL to Treat NSCLC:

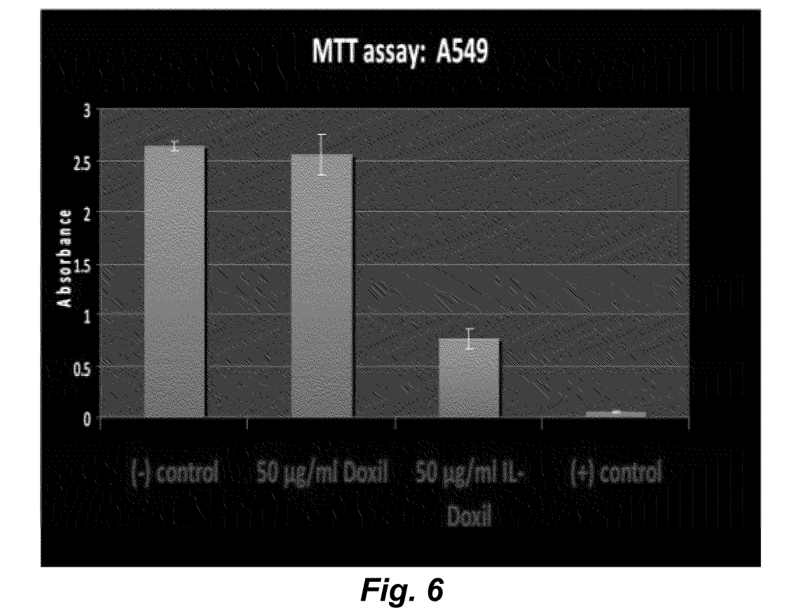

Liposomes are excellent chemotherapy encapsulation vehicles but they can be improved by using mAb to specifically target them to tumors. Specific tumor targeting increases the efficacy of the chemotherapy because a higher concentration of drug localizes to tumor. Tumor-specific targeting spares normal tissue from some of the toxicity associated with chemotherapy. Studies using pegylated liposomal doxorubicin (Doxil) coated with HB22.7 (IL-Doxil) have shown impressive anti-NHL activity (O'Donnell, et al., Invest New Drugs (2010) 28(3):260-7; and Tuscano, et al., Clin Cancer Res. (2010) 16(10):2760-8). Doxorubicin has some efficacy in NSCLC but other agents are considerably more effective. However, as proof of principle, given the availability of Doxil and HB22.7 coated IL-Doxil, the in vitro effects of IL-Doxil on A549 cells was investigated (FIG. 6).

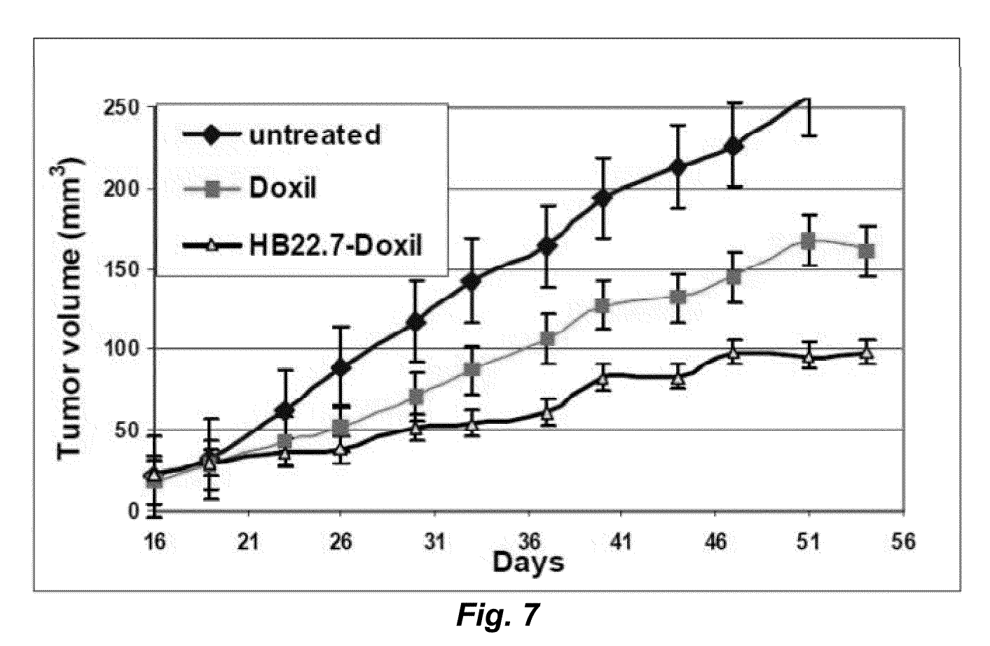

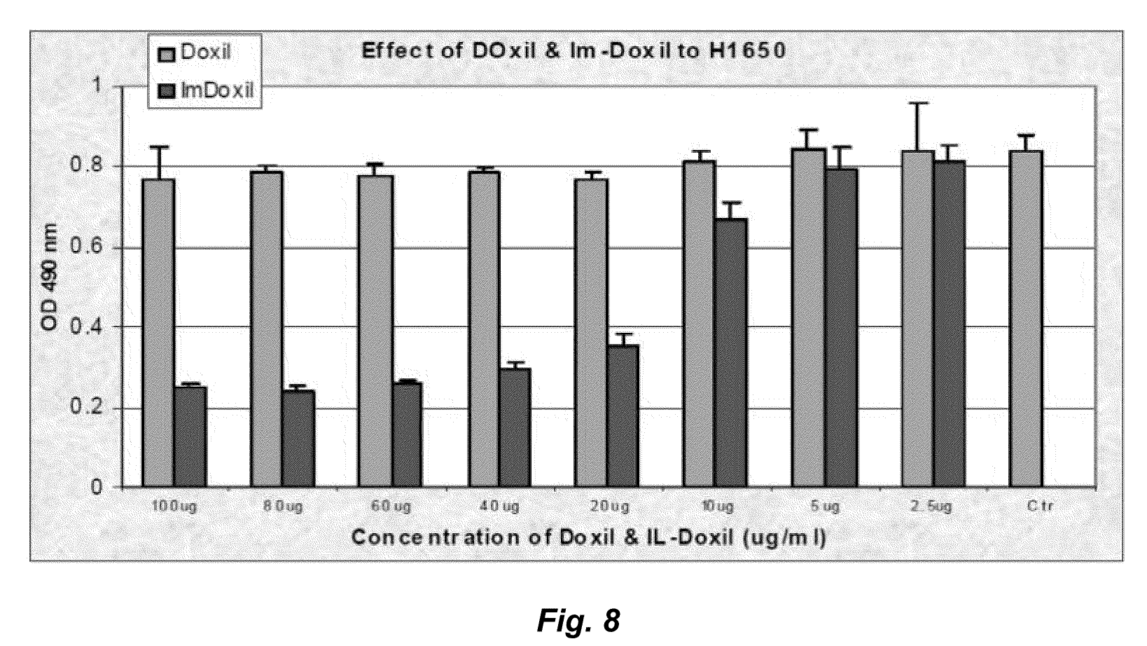

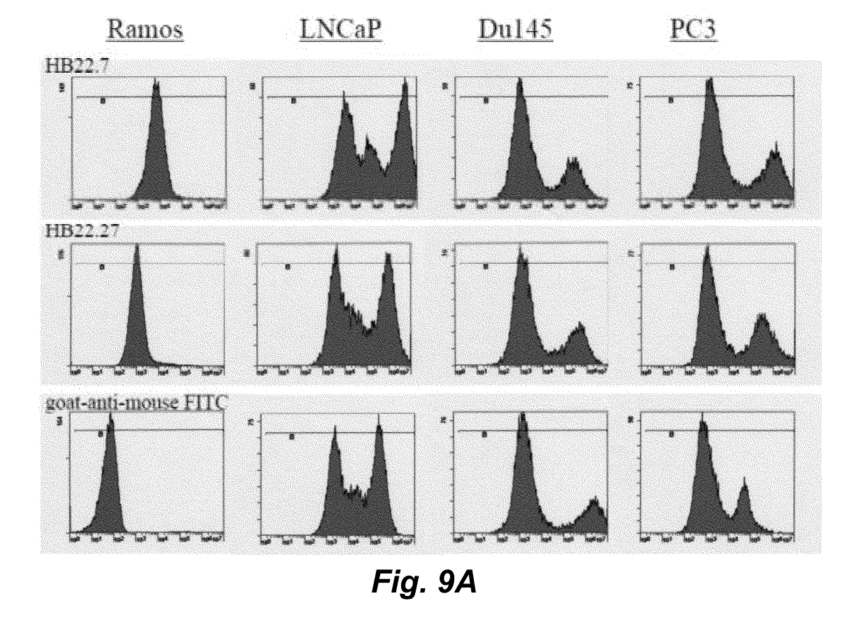

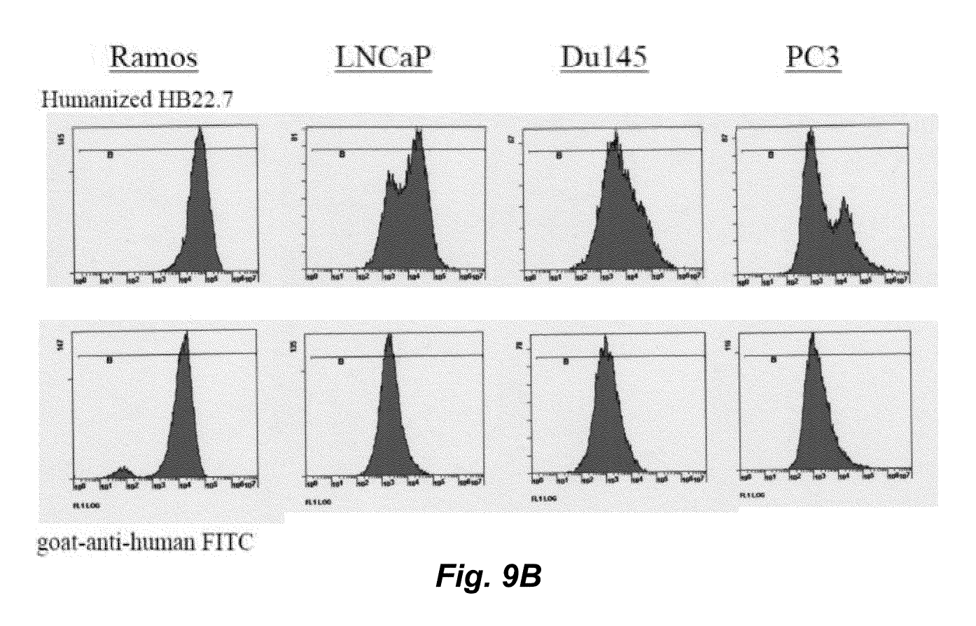

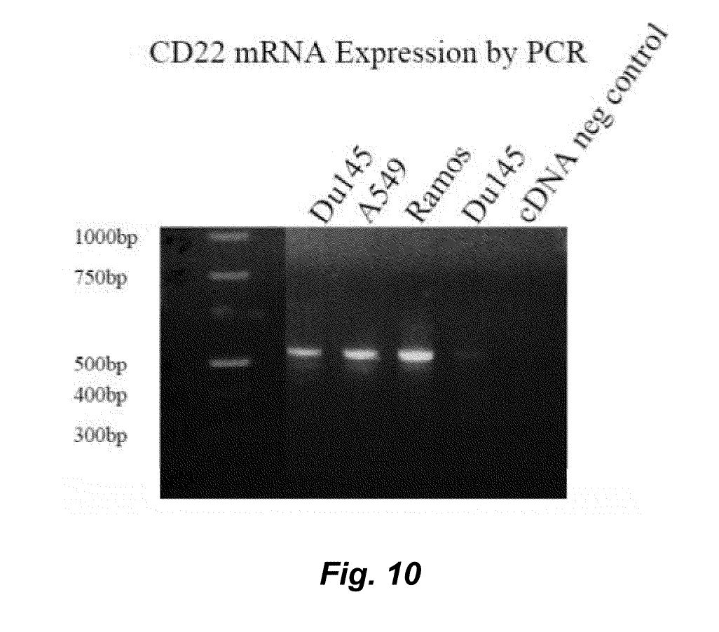

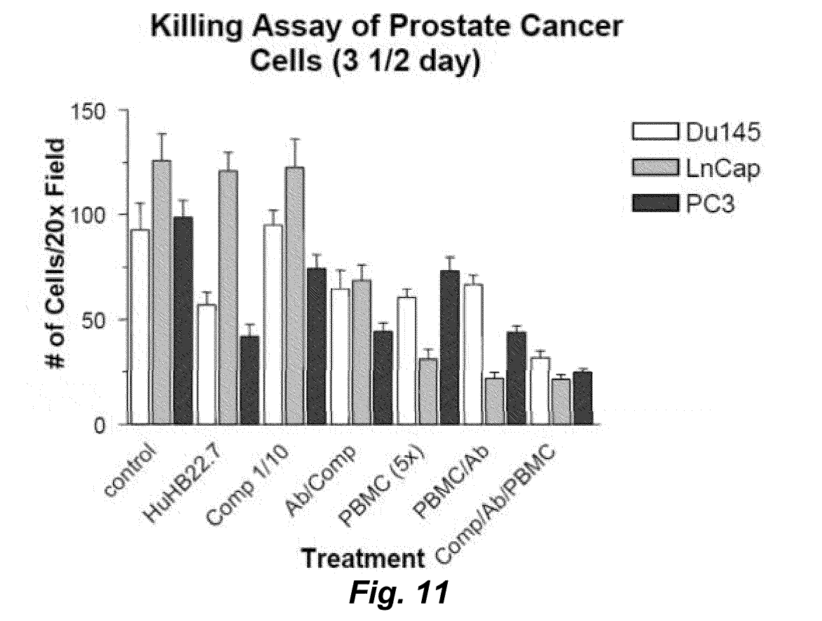

An A549 xenograft trial comparing Doxil to IL-Doxil (FIG. 7) showed significant activity and differences between the non-targeted (Doxil) and targeted (IL-Doxil) drugs. Additional studies examining CD22-targeted IL-Doxil for NSCLC treatment were therefore performed. These data are especially impressive given that Doxil is not the most effective agent for the treatment of NSCLC. Additional in vitro data were generated with IL-Doxil in H1650 lung cancer cells (FIG. 8). These data demonstrate that CD22 can be used as a target for anti-CD22 mAb-bearing payloads.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of preventing, reducing, delaying or inhibiting the proliferation and/or growth of a lung cancer cell comprising contacting the lung cancer cell with an antigen binding molecule that binds to CD22 expressed on the surface of the lung cancer cell.

2. The method of claim 1, wherein the lung cancer cell is a non-small cell lung cancer cell.

3. A method of preventing, reducing, delaying or inhibiting the proliferation and/or growth of a prostate cancer cell comprising contacting the prostate cancer cell with an antigen binding molecule that binds to CD22 expressed on the surface of the prostate cancer cell.

4. The method of claim 1, wherein the antigen binding molecule is an antibody or antibody fragment that binds to CD22.

5. The method of claim 1, wherein the anti-CD22 antibody or antibody fragment is monoclonal.

6. The method of, wherein the anti-CD22 antibody or antibody fragment is a human chimera.

7. The method of claim 1, wherein the anti-CD22 antibody or antibody fragment is humanized.

8. The method of claim 1, wherein the anti-CD22 antibody or antibody fragment is human.

9. The method of claim 1, wherein the anti-CD22 antibody or antibody fragment is HB22.7.

10. The method of claim 1, wherein the antigen binding molecule, or antibody or antibody fragment is conjugated to a therapeutic agent.

11. The method of claim 10, wherein a therapeutic agent is selected from the group consisting of a cytotoxin, a radionuclide, an inhibitory nucleic acid, and an anti-neoplastic agent.

12. The method of claim 10, wherein the therapeutic agent is encapsulated in or on a liposome or in a nanoparticle.

13. The method of claim 1, wherein the lung cancer cell or the prostate cancer cell is in vitro.

14. The method of claim 1, wherein the lung cancer cell or the prostate cancer cell is in vivo.

15. The method of claim 1, wherein the lung cancer cell or the prostate cancer cell is human.

16. A method of preventing, reducing, delaying or inhibiting the proliferation and/or growth and/or metastasis of a lung cancer in a subject in need thereof, comprising administering to the subject an antigen binding molecule that binds to CD22, wherein the antigen binding molecule binds to CD22 expressed on the lung cancer, thereby preventing, reducing, delaying or inhibiting the growth or metastasis of the lung cancer in the subject.

17. The method of claim 16, wherein the lung cancer is non-small cell lung cancer.

18. A method of preventing, reducing, delaying or inhibiting the proliferation and/or growth and/or metastasis of a prostate cancer in a subject in need thereof, comprising administering to the subject an antigen binding molecule that binds to CD22, wherein the antigen binding molecule binds to CD22 expressed on the prostate cancer, thereby preventing, reducing, delaying or inhibiting the growth or metastasis of the prostate cancer in the subject.

19-34. (canceled)
